SEQUENCE LISTING (3 sequences)

SEQ ID NO: 1 - DNA, 16096 bp, Canis familiaris
  mutation (10349)..(10364): 16 bp sequence deleted in DCM-affected dogs
catagtgttatcaaacctatttccaagtgtagatgcaatttagggctgcccccaagttcccacaactgtttaagattgaatagtgataaagcaaggaaggaatcacaaggatacatcagacttggtcccaaccttgatctttttcagggaaagaagttcagagaggagaatacagtaacataaatagctacaatatcatgtcctaggtgaaaaaaggccattaggaagagggatggaggagaggcaatcaggaaagatcgcttgcaacatatacttcgtgccagacagacatttaagagtagaaaagaccatcttaggcaaagatttggattgaggacattctatattttggattgaggtcagagtgaataaattctctgatggtagagggcagggagttaaactgacaaactgatcccttcataactttgcttttaattaacagagtcaaacccaaagaggagactatattactggagaaaaccaaattgatggccttttatggccacagggggtaaaaaaaaaaagtaaatgtatttgggtcagttactcaagcctaagtcttacctaaaggaacctccagttcctgccttgtcctgttcgctcagggagaaaacactagagatggttaggctgcttctctgactgtccaatggcggagaagctcatacctttgaatgcagctgattggctactgtaaaagcccgctctgggcgaggaccaatgacatcccggagttcagattcttggaaatagtttctagtcgggaatgcgtgacattgacacagtctctcgttcgtaaacaaggacgagcctgggcgggcccccagcccacgcgtcccaggcccgggtcccaagcggctaatctcgagctccggtaccccagatgcccgcttcaccttcctccgcccccggctgacccctcacagccccgggccgccgcgccaagtcccgccacccacgggacccgcggccgggggcggggcccaaagttctttatatgaaggcggcccgcttcgtgatgcgcagcgccggctcgctgagcgcggccagcgtggtcccccgcgaggtcgagcttttctcgcgctacagcccgtccccgctgtccatgaagcagctcctggacttcggtgagctggggagggaggtcgggcgggccgccggggctggggagccgccccaactctgagccctcgggcgggggggcggggccaaccgcttaagatacctggacgctgctctcctgctgtcggaaacctgcaggagagccctcaaccctgaaccctcgacgccggggcgccttaccctggcctagagctagctgccttatttctcacagtttgcacctacaggtcaaggccgcttgctgacccactgatgctggatggtttgagctcttatttccgtgcaagcttccagtcaaaacacttttcttgctttgagccaccatatcacaatgaattaaaacctttatcccctccttcacaacattatatactttttttggagaggattcttaatgttataaagcagcctcaaattaagtgagattttatcgcttagccaccttccacttgttgacagggtacattcatctgcattgttaacagttttgtctctttgccagtatgattttctgaatatttgccttactgccagaaatgtttacaaaccttctgcagcggtcttaaaaggaatatttcaagaatttaaatgtgatgatatttatcactaatgtggaaatatcacgattcttacccactgtcttatccttctacttcaatttcgtatctagaaccctctgaacgatgttgtctggtggacattgtggtgttgacagctgtgggacattctgacttaatggaagtgaatcctcttccccccaaaaaggaaacaataattctcatattggtcctttaaaaaaaaaaaaaaagacagtaaagaaaaagaccatgaaggaggagatacttcttattataaagagtaacccaactgagggtgaaactataatttaatgatatgtgtatgtgttcgatattggatatctaatgctactcaagtgtatgattcagatctactttttttcaaaaactaaataaatattatttattacaggtacagaaaatgcatgtgaaagaacttcttttgcatttttgagacaagaattgcctgtgaggctggccaatatcctgaaggaaattgatatcctccctgatcgattagtcaatacctcttcggtgcagttagttaaaagctggtaagtggtgaacccattctgaaacagtctctatttttaagacaagtatctgagtatgaattcttataatacattaacatcatctttggaaatgggtggtctgaacagtaagtatttttgaaagatgtaaagacttttttttagtaagttagggtaagttatcatagggacagttataaagctataaagagacattttgccctgaaaaaggacatactttcatgatataattgtattttaagtaataagagacagaatttgatattgcacatggaatcctattctagaaatagaaggactactatttagaaggtctaatctttgttacttacaaatcaattaacagcaggcgttgggttcaaatgccacctctgcctcttcctagttgtggaattcttagcaagtatcttaaatttgctatgcctcagttcctgcatctgctattgaggaaaataaagtatatgcttcaaagagtttcatgtgagtgaaattaaatgagctaatccattataaagtacttaaaacagtacctagcgcaagccagaacttagtaattgtcagttattgtcatcttattttacaaataaggatcctgaggcttggagagggtagatcacccaaaggaacaaattttcagagaacaggactaggatatgcactgaggcagatattcgccagagcctcaccctaatctactgacagacagctttttgttcattgattctagaaatatatatgtgttataaatattatatagtctataatcaaattataaatattactgtatatattgttaaacagttacaaacacataatccatagtatttgctgctttcacgaattctcttcacctatgaatgtagtaggaaaaattaggaagcttttcttaaaaagctgtttctaggggcagcccaggtggctcagcagtttagcgccgccttcagcccagggcctgatcctggagtcccaggatcgggtcccaagtcgggctccctgcatggagcctgcttctccctctgcctgtgtctctgcctctcccttcctctctctctctctctctctctttctctgtcatgaataaataaaatcttaaaaaaaaaaaaagttggtttctaaatgctataaaaaaagaaaacacttgctagataaaacaaaaactactgactggaatgacttaatctggagtgattaaagaaataaattttaagttagaaatttgagggtcaaagtcatagctttgggtttttttgtttggttggtttggtttgattttagatctaaaaaatgtgctggaggaaagttgtaattttaaacataatgctttctcttttccaatcatttgaaatgatttagtcacttgtatctgacaattcaattttaaatttaaagcacacctgtcactttacaggtatatccagagcctgatggatttggtggaattccatgagaaaagcccagaagaccagaaagcattatcagagtaagttttatgcattagagtgtctaaaactttgactatttccagacatactcctaagattttttttttttagacatgaattaaaggatccacaactctgaataacaaataccctctgccttacaaatgacaagttttgacttgttggttttttaaaagtgatgaagaatgatgacattgaaaatgatgctaagaatgaacaagtgtagatttatttttaaggaaatctatttttagtatacatttagtctgatgactagattcacaggtagatttactatgacatagtatcagtatatgtcattttgtcattggagatagaacattttctttcagattaacttatagtttctccctataagaataggaaagaagaatctttttataaatctgagaagaaatcttgatgtttttttccacaatggcactctagacttatctttaatattaattattttttaattattattttttaaagagaaggcttaatgaattcctagaatgaattcaccagttagaaagctggtatttttatgttaagtgagaagcaaaataaatctgtttgtaatactatctcctctattgtagttggagaatctgtagtgagaaccttaataaaccttcatccaaacattttatcatttgattctttagatgggtacttaaaaaaagcttcagccttctttgaagtaatctaaggcatataagtgaatgaaacttacttgaactttctctaactctaaatgtgttttggcttagttttacagatactctcatcaaagttcgaaatagacactacaatgtagttcctacaatggcacaaggaatcatagaatataaagatggctgtacagttgatccagtcaccaaccaaaatcttcagtatttcttggatcgattttacatgaaccgtatttctacccggatgctgatgaaccagcatagtaagttagctcatcatggtcatgatatactgagatgggtatagaatatcagccataccccaaccagatgtttcattattcaggggttcacattctttttcaatggtagttttaatcattattatcctagtttttatgacatcttttgtttttattcttcaacagttcttatattcagtgactcagagacaggcaacccaactcacattggaagcattgatccaagctgtgatgtggcagccgtggtccaaggtaatttctagatttctgcatttcccttattgaaaatgtacactgtatgattgacatatctatagttgaggtgactttgtgaatgaatttatatccagagtgttccagtttatgtttttataaatactgtcatgaagatcctttgcttttgagagtcaggggtttaaacattcattaataaaattaaaaaatccatacagtaatggccttattgttctatgttctatttaatctttgaagtgtgcatattcctatcgccacccctataacatcaacactgtcttaattttttcttcaataaaatattaaagagatattttttcatacaggtgaatctgtactctaatgaacattctaaattaagggtagtacgcacactgatgatcacatgaaaattttacttggcataaactcatttcatcattatgaaaaaaaaacccttctatttcagatgcctttgagtgttcgaagatgctttgtgaccagtattatttaacgtctccagaattaaagctcacacaagtgaatggtaagcaataaaaaaaaatattttaatctatggaaaattcactgttctcccagtataattacttcttgtaaatacaaagcaactcctgcattccttttcttccttctgaacttgtctagaatttgtatgcttcttttttgaagccaaggttctcttgatctccattgcagagttccagtttgagcatattcagttgctaatgatgattcataaagaggcaaggtttgaacaaacaatttaagatttagagtagcatgagttccagtgtcctagatcaaagaaagatagcttattgcagaatatgagagtcagggaggccagaagtaggaggagccacttggtagcagaaatagcctcctagagaagttactcatgagacctggaaacaaaaacacttcaaattgtactgtaagcaataaggcagtcacaggacaaatactgtgtgattctacttatctgagatatgttaagtagacttagagaggtagagagtagcatggtggtggccagaggctgggggctggggtaaatgggttattttattttattttattttttatttttttaaagattttatttatttactcatgagagacagagagagagagagagagaggcaaagacataggcagagggaaaagcaggctccgtgtagggagccccatgtgggactagatccagggactccaggatcacgcccttgggccaaagataggcgctaaactgctgagccacccagggatccccgatattttattttaaaatgctatttaatggatataaaatttcagttacgcaagattagtaagaaggtagatctcatgttaaatgttcttttaaaaatatttaaaaaaaataaaaaaaataaaaaataaaaaaatatttttaaaaaataaataaaaaataaaaataaatttaaaaaaatatttttaagttaggcagcctgggtggctcagcggtttagcactgccttcagcccagggtgtggtcctggagacctggaatcgagtcccacatggggctccctgcatggagcctgcttctccctctgcctgtgtctctgcgcctctctctctctctctctctctctctctgtgtctctctgtgtgtctcatgaataaataaattaaatctttaaaaaaataaaataaaaaataaataaaaatatttttgaattacaaaaaaaaaatttaactgttctgtaagtatgcaccactgttttggaagcagaccatacttcagtggcttcagatccagggtaactgaaaaaatagggtcttcccacgttgaagtgataatatgatgaatgcaaggcactttttaaatcttctcccatagtgcttctcaaagcaggacagttaaaattaagttgctcagattaaaagtactctaactcgtggattgggaatatgtaaatataattgctaaattaaaaataagaatacaaataaaggttgaaaaagaactctaagcattaacagcattgttgcataatacttgtagataaactgcttgaactgtttgataaaataaggagggaattgggccaggccttctgttttctgattcaaactttgccaaggcctgggacaccaaacaataatacttttatttttcttctcaaacaatattatgaggtttttgtttctgttttcttttgcctctcagtgaaatgtaagcatatttagcccctaagatttaatgtttgggtgataaaaatcaagaattttaaatgtgttattttcccacaaaatatgaaaattatacccaacattaatttcagtattattctatggacacgttaactctttcttttttcttaaaatgtatgattttaactctgaaatacctcagtataaaagttttgaaagcagaaacaaataatccacctaatctaatttatagtaccctgtcacacacttcatctgaccttatacatttgtagagaaatgcaaaaattaatcatagagcacaaggcaacaaccctaatagcattttgaaaataagccagttgtgtttactgtttaaaagaaaagggtctagggtagaatttcagaggaaaggaagatggggaatattctaatctcaggaataaggagtatctattaatcaaatagatattgataaaatattgataaaatatcaaatattttaatgaaatatttaccagttctaagtgagtctaaatttttttctaggaaaatttccaggccagccaattcacattgtatatgtaccttctcaccttcatcatatgctctttgaactattcaaggtatgacacttcacagtgattgcaatttgggttgttttttttttaattcacatgttacatttttatttcctttatcatttcccgctgaatgggagcttcactgtcttctcccaaacacactttagtgctcctaatagaaagaagctatagttaatgtttgaaagggcacatgcagttcattagagcatcaacacatatttgagaatgtgcaatatgattcacgatgtcttaatgctacctgttcttattatttcttcaggtgtccaacaaagcactagtcattcatgcatttgttaaggaaggtcagggggtgaatgcttcagtgaatggggccttgttacctccacccccataggatggtccagctccaaatcccaatgctcaggtccagagtttctgcaggtggcagggctccaacctgagtttagaatatactcagaagtcagtcatttccttccaagatcaacttgaatgtgcatatgaatcacctggggttttgttaaaatgtaaactctgattcagtgtggctgggatgggtcctggggcttagaatttgtaataataagcccataggtgatgctgctggtccacactccacactttccatagcaagactgtgacagtgcttctcaaacttcagtatgcattggaatcacctggagggcttgttaaaaacacttactcttaggctctggcctagagattttgattctgtacatctgagaatttgcatttctaacaagctcacaaaggatgtgaatgttactagtctgggatagccaggtaccaagaagcaaatctgtcctgtgttctaagaccaaactaagactgtacttacaccaaaaaaagaaaaccttctgacacttcatgatgctttccaagagtctcttggactcctgggggaggaagcctaaaaaaagagaaatttacctctaattctgatgtagcttttctcagaatttactatttcctaccttattctaatgttaatgcaatgaggtattgattatactacagacatctgatcttttttcttttacttcagctaatgattagaacttagtattatatagaaaccagaaagcagataatgtaccacaaggaggttagagaggaaaacatgtgaaataaaccaaaactttatctgtgtgagacgcacccagttgtctgaaaacttgtttgagtttcttctctgttaaaggacaaaagatactttttcattgatttcatttatttttaaattgaatggtccaattcctacagttattttgccatcattggtaaaagttaagatgataaccaatgcaaattgaaatttaatttagaattatttactatccttttaaatagtaacaacctttcctggcaagttttataaaagccaataaattggttcaatgattcactgttcatttgttatgtaatagtctttggtgcttggatgtaaaatgtaatggaaagacagaaaagagagagccaggaaatggactataaaaaccaatttctacatttaaattaagcttacaaataagaacttgacagctagcagaattgtttttccattagacattactcccaggagctatattacatggtaaatgaatatttttcattatacatcctgcatgacacattcataaatggctatttacagagctgttttactactaacttaaaaaaaaataggaactttgaattaccctctgtgctgcttttattttaaacataaaaaacagttatttattgtttctcattcttctgccgttgaattccttatggttttgatttctttagaatgcaatgagggcaacagttgaacaccaggaaaactggccttcccttacaccaattgaagtgattgttgtcttgggaaaagaagatcttacaattaaggtaaccattctctgtttttcttttgagtctctctggagaaaactaaaaaagtgtcaataataataaagctctccgctttgagaaacagatcaaagtcatggacatcaggtgacttttaaataaaggaaatatagacaaatgtaccttgtttttaatgcttggctttggtttgtctgtttaagatttcagacagaggaggtggtgttcccctgaggatcattgatcgcctctttagttacacatactccacggcaccaacgcctgtgatggataattcccggaatgctcctttggtaagaccaattagatggccaagttcaccccagccacctagttataaatgtaaagcaaaggagtacaaggaagtatttagtcaagctactcaatttagttaaattgggcagggggtagggggtatgagaaagcatgataaagcaagctgaagacagtgaaacaattctatgagaggtctctgattggaagtagaaaatacttatctgggttcaagaacatcagcagcagggaggataataactccttcttacattttgtatttcaatgatcatgtccacttctttgccagtaactgatcttttccctctaggctggttttggttatggcttaccaatttctcgtctctatgccaagtactttcaaggagatctgaatctctactctatgtcaggatatggaacagatgccgtcatctacttaaaggtatcctttcaacccagtagaaataataatatttctatttttaagatttaattaattaattaatttatttataaatttatttgagagagagagagtccatgcaagtgggggaaggggcacagggagagagagagaaagagaactcttcttggctgagcatggagcctgatgtagggcttgatctcacaagcctgagatcatgacctgagccaaaatcaagagtcaggcacccaaccaaccaagccacccaagatgccctgaaacagtaattctgaaagcagttcttttccggacctcagttccatggtccatagtgaatacctcaaactaaactaatgcaaataatgaccaaaagtcatttcattttctttttttttttttaatttttatttatttatgatagtcacagagagagagaggcagagacatagacagagggagaagcaggctccatgcaccgggagcccgacatgggattcgatcccgggtctccaggatcgcgccctgggccaaagtcaggcaccaaaccactgcgccacccagggatccccatttcattttctgtttgatatgcatagacatactatttaataatatttaatatggttacttaccaatcggaagtggggccaggacacctgcttcaagcgaagccaaattgtttctcataaaacatattagtttccacataaggagatgttcacgtgaattttttaaaccaacaccttttggcatttgtgagaatagctgtaccatcattttatttaaactcatgggaaatagatgctaattatgttccagcctagaaaacaactgataccaagatacttcagaccttcagctgaccttagtagaaatccagggaaggaaaagctaactgctcacaagctagcaatgggacaatctgacatttcctagttaaactttaaaatttggtacatggtttttctatttctgataaaagcttcaatgtgatttgttaaggtcactgtcctgatttttcatcattttgcagctgttttgactgaaatgtatttacagacattagtatctaaaatgtgaagctcgtaggctctcattgtgtgcaaaggaattatttgctcagaggccttggactatctgaaatagttaacgtattcatcttatagaaagtcttcgaagtactgggaaagattaagagatcacagatttctattatcagactttaataaattaatagcatcagattagaactctatatcacagatacaaaggtttttgtaaataaagaatagctttgttcttcagttctatcacatcagcaatatccaaacaagggacctgtgtaaacattgaagacaaattctgttcctcaatttatagttgtatatatactatctcagcctctagagggtagaaataatatttggttttagtttttttttttttttttatacactgtctagcgtaccatcagctatgtatggtgtgctcttcttattgaatcaattaaagggatgtatttgaatgtttgaatactttcttctgaatgatcacatcaagtacaaattatgccatgaagaatttctttgggttgatttttttcttccctctcttcttttcaggctttatcttcagagtctgtagaaaaactcccagtctttaacaaatcagccttcaaacattatcaaatgagcatcgaggccgatgactggtgtatcccaagcaaggaaccaaagaatctggcaaaggggaaagtggctctgtgaaagtggacactcaggacactttaagggatcaaagtgggtctaagccagtgctgcttcctgaatgtttgtgtgtgaactcgtttcctcaaaaacaaacaaaagcaacaaagactccttgttgggaacagtggtgcaagaaggaacgaagcccgttcctcggacccaggagagtgtaatgtaggtgttaacagatggccagctctctggtctttaaacttagcataacgtacaagcttctatcagatcctgaagcaggaaaagcttcagtttgccctgtctttatcgacaagaatgggaacgaagtaaagatataagctagcagtttcaatagactgatattttaggcctgtgattgatggcatgagataatggtatttattgcatatcgagtgtcaatatgccagtatttccctgacatcaaagggttagggcttactttatgctctgtaattagaaatgtatgtaaaggtacatttcttgtatttctgtgttctctagggaggaaaagctggtgactaaataacatttactttgtatgttatttgagtgagggaggaatgagtcaaaacgttttggaaattcagaagaaagcctcttctgataaataccgtatcttcattatgctgctttcctgtttacacttcatataataagtgaacacttattgccaggtgcctgcctagtcttgggtgagttttaaaatatgcactgaattgaggatgttggcagttggagggagagaagaattgccaaagtgatagcaaaactctcttcgccttactttgtttataaatgaaatgtacattggaaaaaccaatgttagggaaagaaactatacattcagaacctaactcagtaggaggggctgttcactcccctgcaatggtggtaatccaatgatggcattccttttctaggcttaaaaaggtatatttttgatacatgtaattatattctctacactcctgcattaacatgtccataaaaataattcctaattatcaagtgattcaagaacattcaaacttaaatacttcttgggatatttctaacaaacagcctgggagtaagaggttttcattccaggttgctttcatttgaatcattgagactgagggatgataaatgaaagttgtaatgcgtatggaacttttggggactttgatataatctgtaggctgacaggccggtctagcacatcactctcacagaaggaagagaaaaacagaaagaaggaaggaagagggaagaaagaaacagaagacaggcatcttgcttccccccccacccccacaaattattttaagaaatgatctatattttcatagctaaattactggcaagaggatgaattggtctttaaaaacagaatggcttctttaaatagttccctcacttttgcaaggtaatagtcttttatgagataatctcatctgtgacaaattggtaggtaaattgggatatagaaaaaacctagaattttctgcattgtaaaataataagtactgtttatgtttgtatatgtagaatttaacatttgtgtttaattttgctgtggtctagaattgtggtgtttctgtagtggccataaaggcctgaaatgatttattatggatgtcagactttatcatgagaagtcctaggtaatgacttttcttgatggtgggactctgactcatgcatttatcaattacacaaaattatttgcaatagattatttgacttttcagtttttactgaaattttaaacattttgcatgtaaatacttgtatttaccaaagatttaaagcagttgattaattaatccaaacactgtgaactgtctttgaaatactagagaaaaagaaatgttaatatcacagtaacatcaactatactgtataaatagagttctataatacagaaatagtctacattggtatttgtgaaatctatggaagaactttaggattctagcagatggatactgaatgttcaggcccacttaagttattgatgtataaattgtgtttttgtctctatgtacagagatgtaaccatttttataataaatatttttattacaacaggaaacatagtcttttgaaattcatttctcaaatagatggcaaattactaagaaaaatttgaggtaaacattttcctcactttacatataaatctcattcatctatatattgggtatagccttttataaaagtattaaaagcaacttaaaattttgatttttaaacaaaagataagtactttatagcagaaatttgcagttcttctttgctttcttgtgtatgcttcaatagcacatcacatagtacacagtgtattaatctacatgaatatattgaatatattccataatcatttttgcaatgtatacattctgtacatttttggggtaaactttaagtttcccttggtgattgtacttacataacaaatatagtaaagtaccaaaataattgcctagataatttaggttcccaaacaacttctggattaaatatctagataataataatgtagaaataagaaattataaattaatcttcacattgtctttcttaactaacttagtcctttgtctttagataacatcctttgcctaacttcacaatgcccttattggcaaagttttgtaaacaaaaaaaagaaaaaaaaagaggggggagaggaaagaaagaaaaagaggaagaaaaaattagtagaatagatttgtcataactaaattctaaaatcttgtcagaataagcatattggaatcagaaatgccatcaacctaaggcattagacccaaaaaaaacatacaggaaagccctttatcaatgaccgtctcctttatttactaccttactttctttcatcagggaaatactgcaccatgtgtctaaagaaagcgcatgcacacatgcatacacatgcaaacacgtacacatgtacctggaacttagctgtacagtgtccttagactaagctgcctggaaagtagagagttagtgcactgtataggctcagactcttcatccacattttcctaaaaactgcaggacttcaagtcccacaattccccacatatgcacacacacatctttttgactcaagtcaaaggaaactaatttccaagttaaacaaagttggctgcattttcatcaaactatcattataccatttctcccaaatttcaaatttggctagcaatggcaaaagcatttgatcttaataactacagtgacatgtacagtgagagaggaaagaaaaagagatctttgaaagctagtagcttaggcattgaccttgataatttcttcattctcagctatactttcaagtagtgcacttaggcttgactgtgtaccaacactgcaaatgtacatgaaccctgtagatcactttgatttggcctggtgaaagaattgagggactttcataatttcacaagagcttagataagaagtccccaccccccccatcccccgctgtgaacacggagagcagtgttccattaatgtgttatccatacacctctctttttgatttttcactttttaccttccatcacagttacttaggcagtccatagacattcactggacagatacagaaataaacacataaataaactgcctatagaacagcatctgcatttaaggggagattagactctgtgcacatcaaactttacacaattttatgctttaaatggtattaaagaagttttacaatgttgcagaaactctggaatacttatccaagcaagttaaattgcaactgatctttgtttcatgttactctctgttggtggtctttggccattggaaattatcacatatttcgagagaaatgaattaacaggtaaagttattaatactaaggaaatctcttgagcaaatgggtcagtgtttcagatctgtgagtattctttcttcagatgaaaacatatccccatgcccatttcatggccctttaatggaaatgagcaactgtcttttaatgagatctttc

SEQ ID NO: 2 - DNA, 16080 bp, Canis familiaris
catagtgttatcaaacctatttccaagtgtagatgcaatttagggctgcccccaagttcccacaactgtttaagattgaatagtgataaagcaaggaaggaatcacaaggatacatcagacttggtcccaaccttgatctttttcagggaaagaagttcagagaggagaatacagtaacataaatagctacaatatcatgtcctaggtgaaaaaaggccattaggaagagggatggaggagaggcaatcaggaaagatcgcttgcaacatatacttcgtgccagacagacatttaagagtagaaaagaccatcttaggcaaagatttggattgaggacattctatattttggattgaggtcagagtgaataaattctctgatggtagagggcagggagttaaactgacaaactgatcccttcataactttgcttttaattaacagagtcaaacccaaagaggagactatattactggagaaaaccaaattgatggccttttatggccacagggggtaaaaaaaaaaagtaaatgtatttgggtcagttactcaagcctaagtcttacctaaaggaacctccagttcctgccttgtcctgttcgctcagggagaaaacactagagatggttaggctgcttctctgactgtccaatggcggagaagctcatacctttgaatgcagctgattggctactgtaaaagcccgctctgggcgaggaccaatgacatcccggagttcagattcttggaaatagtttctagtcgggaatgcgtgacattgacacagtctctcgttcgtaaacaaggacgagcctgggcgggcccccagcccacgcgtcccaggcccgggtcccaagcggctaatctcgagctccggtaccccagatgcccgcttcaccttcctccgcccccggctgacccctcacagccccgggccgccgcgccaagtcccgccacccacgggacccgcggccgggggcggggcccaaagttctttatatgaaggcggcccgcttcgtgatgcgcagcgccggctcgctgagcgcggccagcgtggtcccccgcgaggtcgagcttttctcgcgctacagcccgtccccgctgtccatgaagcagctcctggacttcggtgagctggggagggaggtcgggcgggccgccggggctggggagccgccccaactctgagccctcgggcgggggggcggggccaaccgcttaagatacctggacgctgctctcctgctgtcggaaacctgcaggagagccctcaaccctgaaccctcgacgccggggcgccttaccctggcctagagctagctgccttatttctcacagtttgcacctacaggtcaaggccgcttgctgacccactgatgctggatggtttgagctcttatttccgtgcaagcttccagtcaaaacacttttcttgctttgagccaccatatcacaatgaattaaaacctttatcccctccttcacaacattatatactttttttggagaggattcttaatgttataaagcagcctcaaattaagtgagattttatcgcttagccaccttccacttgttgacagggtacattcatctgcattgttaacagttttgtctctttgccagtatgattttctgaatatttgccttactgccagaaatgtttacaaaccttctgcagcggtcttaaaaggaatatttcaagaatttaaatgtgatgatatttatcactaatgtggaaatatcacgattcttacccactgtcttatccttctacttcaatttcgtatctagaaccctctgaacgatgttgtctggtggacattgtggtgttgacagctgtgggacattctgacttaatggaagtgaatcctcttccccccaaaaaggaaacaataattctcatattggtcctttaaaaaaaaaaaaaaagacagtaaagaaaaagaccatgaaggaggagatacttcttattataaagagtaacccaactgagggtgaaactataatttaatgatatgtgtatgtgttcgatattggatatctaatgctactcaagtgtatgattcagatctactttttttcaaaaactaaataaatattatttattacaggtacagaaaatgcatgtgaaagaacttcttttgcatttttgagacaagaattgcctgtgaggctggccaatatcctgaaggaaattgatatcctccctgatcgattagtcaatacctcttcggtgcagttagttaaaagctggtaagtggtgaacccattctgaaacagtctctatttttaagacaagtatctgagtatgaattcttataatacattaacatcatctttggaaatgggtggtctgaacagtaagtatttttgaaagatgtaaagacttttttttagtaagttagggtaagttatcatagggacagttataaagctataaagagacattttgccctgaaaaaggacatactttcatgatataattgtattttaagtaataagagacagaatttgatattgcacatggaatcctattctagaaatagaaggactactatttagaaggtctaatctttgttacttacaaatcaattaacagcaggcgttgggttcaaatgccacctctgcctcttcctagttgtggaattcttagcaagtatcttaaatttgctatgcctcagttcctgcatctgctattgaggaaaataaagtatatgcttcaaagagtttcatgtgagtgaaattaaatgagctaatccattataaagtacttaaaacagtacctagcgcaagccagaacttagtaattgtcagttattgtcatcttattttacaaataaggatcctgaggcttggagagggtagatcacccaaaggaacaaattttcagagaacaggactaggatatgcactgaggcagatattcgccagagcctcaccctaatctactgacagacagctttttgttcattgattctagaaatatatatgtgttataaatattatatagtctataatcaaattataaatattactgtatatattgttaaacagttacaaacacataatccatagtatttgctgctttcacgaattctcttcacctatgaatgtagtaggaaaaattaggaagcttttcttaaaaagctgtttctaggggcagcccaggtggctcagcagtttagcgccgccttcagcccagggcctgatcctggagtcccaggatcgggtcccaagtcgggctccctgcatggagcctgcttctccctctgcctgtgtctctgcctctcccttcctctctctctctctctctctctttctctgtcatgaataaataaaatcttaaaaaaaaaaaaagttggtttctaaatgctataaaaaaagaaaacacttgctagataaaacaaaaactactgactggaatgacttaatctggagtgattaaagaaataaattttaagttagaaatttgagggtcaaagtcatagctttgggtttttttgtttggttggtttggtttgattttagatctaaaaaatgtgctggaggaaagttgtaattttaaacataatgctttctcttttccaatcatttgaaatgatttagtcacttgtatctgacaattcaattttaaatttaaagcacacctgtcactttacaggtatatccagagcctgatggatttggtggaattccatgagaaaagcccagaagaccagaaagcattatcagagtaagttttatgcattagagtgtctaaaactttgactatttccagacatactcctaagattttttttttttagacatgaattaaaggatccacaactctgaataacaaataccctctgccttacaaatgacaagttttgacttgttggttttttaaaagtgatgaagaatgatgacattgaaaatgatgctaagaatgaacaagtgtagatttatttttaaggaaatctatttttagtatacatttagtctgatgactagattcacaggtagatttactatgacatagtatcagtatatgtcattttgtcattggagatagaacattttctttcagattaacttatagtttctccctataagaataggaaagaagaatctttttataaatctgagaagaaatcttgatgtttttttccacaatggcactctagacttatctttaatattaattattttttaattattattttttaaagagaaggcttaatgaattcctagaatgaattcaccagttagaaagctggtatttttatgttaagtgagaagcaaaataaatctgtttgtaatactatctcctctattgtagttggagaatctgtagtgagaaccttaataaaccttcatccaaacattttatcatttgattctttagatgggtacttaaaaaaagcttcagccttctttgaagtaatctaaggcatataagtgaatgaaacttacttgaactttctctaactctaaatgtgttttggcttagttttacagatactctcatcaaagttcgaaatagacactacaatgtagttcctacaatggcacaaggaatcatagaatataaagatggctgtacagttgatccagtcaccaaccaaaatcttcagtatttcttggatcgattttacatgaaccgtatttctacccggatgctgatgaaccagcatagtaagttagctcatcatggtcatgatatactgagatgggtatagaatatcagccataccccaaccagatgtttcattattcaggggttcacattctttttcaatggtagttttaatcattattatcctagtttttatgacatcttttgtttttattcttcaacagttcttatattcagtgactcagagacaggcaacccaactcacattggaagcattgatccaagctgtgatgtggcagccgtggtccaaggtaatttctagatttctgcatttcccttattgaaaatgtacactgtatgattgacatatctatagttgaggtgactttgtgaatgaatttatatccagagtgttccagtttatgtttttataaatactgtcatgaagatcctttgcttttgagagtcaggggtttaaacattcattaataaaattaaaaaatccatacagtaatggccttattgttctatgttctatttaatctttgaagtgtgcatattcctatcgccacccctataacatcaacactgtcttaattttttcttcaataaaatattaaagagatattttttcatacaggtgaatctgtactctaatgaacattctaaattaagggtagtacgcacactgatgatcacatgaaaattttacttggcataaactcatttcatcattatgaaaaaaaaacccttctatttcagatgcctttgagtgttcgaagatgctttgtgaccagtattatttaacgtctccagaattaaagctcacacaagtgaatggtaagcaataaaaaaaaatattttaatctatggaaaattcactgttctcccagtataattacttcttgtaaatacaaagcaactcctgcattccttttcttccttctgaacttgtctagaatttgtatgcttcttttttgaagccaaggttctcttgatctccattgcagagttccagtttgagcatattcagttgctaatgatgattcataaagaggcaaggtttgaacaaacaatttaagatttagagtagcatgagttccagtgtcctagatcaaagaaagatagcttattgcagaatatgagagtcagggaggccagaagtaggaggagccacttggtagcagaaatagcctcctagagaagttactcatgagacctggaaacaaaaacacttcaaattgtactgtaagcaataaggcagtcacaggacaaatactgtgtgattctacttatctgagatatgttaagtagacttagagaggtagagagtagcatggtggtggccagaggctgggggctggggtaaatgggttattttattttattttattttttatttttttaaagattttatttatttactcatgagagacagagagagagagagagagaggcaaagacataggcagagggaaaagcaggctccgtgtagggagccccatgtgggactagatccagggactccaggatcacgcccttgggccaaagataggcgctaaactgctgagccacccagggatccccgatattttattttaaaatgctatttaatggatataaaatttcagttacgcaagattagtaagaaggtagatctcatgttaaatgttcttttaaaaatatttaaaaaaaataaaaaaaataaaaaataaaaaaatatttttaaaaaataaataaaaaataaaaataaatttaaaaaaatatttttaagttaggcagcctgggtggctcagcggtttagcactgccttcagcccagggtgtggtcctggagacctggaatcgagtcccacatggggctccctgcatggagcctgcttctccctctgcctgtgtctctgcgcctctctctctctctctctctctctctctgtgtctctctgtgtgtctcatgaataaataaattaaatctttaaaaaaataaaataaaaaataaataaaaatatttttgaattacaaaaaaaaaatttaactgttctgtaagtatgcaccactgttttggaagcagaccatacttcagtggcttcagatccagggtaactgaaaaaatagggtcttcccacgttgaagtgataatatgatgaatgcaaggcactttttaaatcttctcccatagtgcttctcaaagcaggacagttaaaattaagttgctcagattaaaagtactctaactcgtggattgggaatatgtaaatataattgctaaattaaaaataagaatacaaataaaggttgaaaaagaactctaagcattaacagcattgttgcataatacttgtagataaactgcttgaactgtttgataaaataaggagggaattgggccaggccttctgttttctgattcaaactttgccaaggcctgggacaccaaacaataatacttttatttttcttctcaaacaatattatgaggtttttgtttctgttttcttttgcctctcagtgaaatgtaagcatatttagcccctaagatttaatgtttgggtgataaaaatcaagaattttaaatgtgttattttcccacaaaatatgaaaattatacccaacattaatttcagtattattctatggacacgttaactctttcttttttcttaaaatgtatgattttaactctgaaatacctcagtataaaagttttgaaagcagaaacaaataatccacctaatctaatttatagtaccctgtcacacacttcatctgaccttatacatttgtagagaaatgcaaaaattaatcatagagcacaaggcaacaaccctaatagcattttgaaaataagccagttgtgtttactgtttaaaagaaaagggtctagggtagaatttcagaggaaaggaagatggggaatattctaatctcaggaataaggagtatctattaatcaaatagatattgataaaatattgataaaatatcaaatattttaatgaaatatttaccagttctaagtgagtctaaatttttttctaggaaaatttccaggccagccaattcacattgtatatgtaccttctcaccttcatcatatgctctttgaactattcaaggtatgacacttcacagtgattgcaatttgggttgttttttttttaattcacatgttacatttttatttcctttatcatttcccgctgaatgggagcttcactgtcttctcccaaacacactttagtgctcctaatagaaagaagctatagttaatgtttgaaagggcacatgcagttcattagagcatcaacacatatttgagaatgtgcaatatgattcacgatgtcttaatgctacctgttcttattatttcttcaggtgtccaacaaagcactagtcattcatgcatttgttaaggaaggtcagggggtgaatgcttcagtgaatggggccttgttacctccacccccataggatggtccagctccaaatcccaatgctcaggtccagagtttctgcaggtggcagggctccaacctgagtttagaatatactcagaagtcagtcatttccttccaagatcaacttgaatgtgcatatgaatcacctggggttttgttaaaatgtaaactctgattcagtgtggctgggatgggtcctggggcttagaatttgtaataataagcccataggtgatgctgctggtccacactccacactttccatagcaagactgtgacagtgcttctcaaacttcagtatgcattggaatcacctggagggcttgttaaaaacacttactcttaggctctggcctagagattttgattctgtacatctgagaatttgcatttctaacaagctcacaaaggatgtgaatgttactagtctgggatagccaggtaccaagaagcaaatctgtcctgtgttctaagaccaaactaagactgtacttacaccaaaaaaagaaaaccttctgacacttcatgatgctttccaagagtctcttggactcctgggggaggaagcctaaaaaaagagaaatttacctctaattctgatgtagcttttctcagaatttactatttcctaccttattctaatgttaatgcaatgaggtattgattatactacagacatctgatcttttttcttttacttcagctaatgattagaacttagtattatatagaaaccagaaagcagataatgtaccacaaggaggttagagaggaaaacatgtgaaataaaccaaaactttatctgtgtgagacgcacccagttgtctgaaaacttgtttgagtttcttctctgttaaaggacaaaagatactttttcattgatttcatttatttttaaattgaatggtccaattcctacagttattttgccatcattggtaaaagttaagatgataaccaatgcaaattgaaatttaatttagaattatttactatccttttaaatagtaacaacctttcctggcaagttttataaaagccaataaattggttcaatgattcactgttcatttgttatgtaatagtctttggtgcttggatgtaaaatgtaatggaaagacagaaaagagagagccaggaaatggactataaaaaccaatttctacatttaaattaagcttacaaataagaacttgacagctagcagaattgtttttccattagacattactcccaggagctatattacatggtaaatgaatatttttcattatacatcctgcatgacacattcataaatggctatttacagagctgttttactactaacttaaaaaaaaataggaactttgaattaccctctgtgctgcttttattttaaacataaaaaacagttatttattgtttctcattcttctgccgttgaattccttatggttttgatttctttagaatgcaatgagggcaacagttgaacaccaggaaaactggccttcccttacaccaattgaagtgattgttgtcttgggaaaagaagatcttacaattaaggtaaccattctctgtttttcttttgagtctctctggagaaaactaaaaaagtgtcaataataataaagctctccgctttgagaaacagatcaaagtcatggacatcaggtgacttttaaataaaggaaatatagacaaatgtaccttgtttttaatgcttggctttggtttgtctgtttaagatttcagacagaggaggtggtgttcccctgaggatcattgatcgcctctttagttacacatactccacggcaccaacgcctgtgatggataattcccggaatgctcctttggtaagaccaattagatggccaagttcaccccagccacctagttataaatgtaaagcaaaggagtacaaggaagtatttagtcaagctactcaatttagttaaattgggcagggggtagggggtatgagaaagcatgataaagcaagctgaagacagtgaaacaattctatgagaggtctctgattggaagtagaaaatacttatctgggttcaagaacatcagcagcagggaggataataactccttcttacattttgtatttcaatgatcatgtccacttctttgccagtaactgatcttttccctctaggctggttttggttatggcttaccaatttctcgtctctatgccaagtactttcaaggagatctgaatctctactctatgtcaggatatggaacagatgccgtcatctacttaaaggtagaaataataatatttctatttttaagatttaattaattaattaatttatttataaatttatttgagagagagagagtccatgcaagtgggggaaggggcacagggagagagagagaaagagaactcttcttggctgagcatggagcctgatgtagggcttgatctcacaagcctgagatcatgacctgagccaaaatcaagagtcaggcacccaaccaaccaagccacccaagatgccctgaaacagtaattctgaaagcagttcttttccggacctcagttccatggtccatagtgaatacctcaaactaaactaatgcaaataatgaccaaaagtcatttcattttctttttttttttttaatttttatttatttatgatagtcacagagagagagaggcagagacatagacagagggagaagcaggctccatgcaccgggagcccgacatgggattcgatcccgggtctccaggatcgcgccctgggccaaagtcaggcaccaaaccactgcgccacccagggatccccatttcattttctgtttgatatgcatagacatactatttaataatatttaatatggttacttaccaatcggaagtggggccaggacacctgcttcaagcgaagccaaattgtttctcataaaacatattagtttccacataaggagatgttcacgtgaattttttaaaccaacaccttttggcatttgtgagaatagctgtaccatcattttatttaaactcatgggaaatagatgctaattatgttccagcctagaaaacaactgataccaagatacttcagaccttcagctgaccttagtagaaatccagggaaggaaaagctaactgctcacaagctagcaatgggacaatctgacatttcctagttaaactttaaaatttggtacatggtttttctatttctgataaaagcttcaatgtgatttgttaaggtcactgtcctgatttttcatcattttgcagctgttttgactgaaatgtatttacagacattagtatctaaaatgtgaagctcgtaggctctcattgtgtgcaaaggaattatttgctcagaggccttggactatctgaaatagttaacgtattcatcttatagaaagtcttcgaagtactgggaaagattaagagatcacagatttctattatcagactttaataaattaatagcatcagattagaactctatatcacagatacaaaggtttttgtaaataaagaatagctttgttcttcagttctatcacatcagcaatatccaaacaagggacctgtgtaaacattgaagacaaattctgttcctcaatttatagttgtatatatactatctcagcctctagagggtagaaataatatttggttttagtttttttttttttttttatacactgtctagcgtaccatcagctatgtatggtgtgctcttcttattgaatcaattaaagggatgtatttgaatgtttgaatactttcttctgaatgatcacatcaagtacaaattatgccatgaagaatttctttgggttgatttttttcttccctctcttcttttcaggctttatcttcagagtctgtagaaaaactcccagtctttaacaaatcagccttcaaacattatcaaatgagcatcgaggccgatgactggtgtatcccaagcaaggaaccaaagaatctggcaaaggggaaagtggctctgtgaaagtggacactcaggacactttaagggatcaaagtgggtctaagccagtgctgcttcctgaatgtttgtgtgtgaactcgtttcctcaaaaacaaacaaaagcaacaaagactccttgttgggaacagtggtgcaagaaggaacgaagcccgttcctcggacccaggagagtgtaatgtaggtgttaacagatggccagctctctggtctttaaacttagcataacgtacaagcttctatcagatcctgaagcaggaaaagcttcagtttgccctgtctttatcgacaagaatgggaacgaagtaaagatataagctagcagtttcaatagactgatattttaggcctgtgattgatggcatgagataatggtatttattgcatatcgagtgtcaatatgccagtatttccctgacatcaaagggttagggcttactttatgctctgtaattagaaatgtatgtaaaggtacatttcttgtatttctgtgttctctagggaggaaaagctggtgactaaataacatttactttgtatgttatttgagtgagggaggaatgagtcaaaacgttttggaaattcagaagaaagcctcttctgataaataccgtatcttcattatgctgctttcctgtttacacttcatataataagtgaacacttattgccaggtgcctgcctagtcttgggtgagttttaaaatatgcactgaattgaggatgttggcagttggagggagagaagaattgccaaagtgatagcaaaactctcttcgccttactttgtttataaatgaaatgtacattggaaaaaccaatgttagggaaagaaactatacattcagaacctaactcagtaggaggggctgttcactcccctgcaatggtggtaatccaatgatggcattccttttctaggcttaaaaaggtatatttttgatacatgtaattatattctctacactcctgcattaacatgtccataaaaataattcctaattatcaagtgattcaagaacattcaaacttaaatacttcttgggatatttctaacaaacagcctgggagtaagaggttttcattccaggttgctttcatttgaatcattgagactgagggatgataaatgaaagttgtaatgcgtatggaacttttggggactttgatataatctgtaggctgacaggccggtctagcacatcactctcacagaaggaagagaaaaacagaaagaaggaaggaagagggaagaaagaaacagaagacaggcatcttgcttccccccccacccccacaaattattttaagaaatgatctatattttcatagctaaattactggcaagaggatgaattggtctttaaaaacagaatggcttctttaaatagttccctcacttttgcaaggtaatagtcttttatgagataatctcatctgtgacaaattggtaggtaaattgggatatagaaaaaacctagaattttctgcattgtaaaataataagtactgtttatgtttgtatatgtagaatttaacatttgtgtttaattttgctgtggtctagaattgtggtgtttctgtagtggccataaaggcctgaaatgatttattatggatgtcagactttatcatgagaagtcctaggtaatgacttttcttgatggtgggactctgactcatgcatttatcaattacacaaaattatttgcaatagattatttgacttttcagtttttactgaaattttaaacattttgcatgtaaatacttgtatttaccaaagatttaaagcagttgattaattaatccaaacactgtgaactgtctttgaaatactagagaaaaagaaatgttaatatcacagtaacatcaactatactgtataaatagagttctataatacagaaatagtctacattggtatttgtgaaatctatggaagaactttaggattctagcagatggatactgaatgttcaggcccacttaagttattgatgtataaattgtgtttttgtctctatgtacagagatgtaaccatttttataataaatatttttattacaacaggaaacatagtcttttgaaattcatttctcaaatagatggcaaattactaagaaaaatttgaggtaaacattttcctcactttacatataaatctcattcatctatatattgggtatagccttttataaaagtattaaaagcaacttaaaattttgatttttaaacaaaagataagtactttatagcagaaatttgcagttcttctttgctttcttgtgtatgcttcaatagcacatcacatagtacacagtgtattaatctacatgaatatattgaatatattccataatcatttttgcaatgtatacattctgtacatttttggggtaaactttaagtttcccttggtgattgtacttacataacaaatatagtaaagtaccaaaataattgcctagataatttaggttcccaaacaacttctggattaaatatctagataataataatgtagaaataagaaattataaattaatcttcacattgtctttcttaactaacttagtcctttgtctttagataacatcctttgcctaacttcacaatgcccttattggcaaagttttgtaaacaaaaaaaagaaaaaaaaagaggggggagaggaaagaaagaaaaagaggaagaaaaaattagtagaatagatttgtcataactaaattctaaaatcttgtcagaataagcatattggaatcagaaatgccatcaacctaaggcattagacccaaaaaaaacatacaggaaagccctttatcaatgaccgtctcctttatttactaccttactttctttcatcagggaaatactgcaccatgtgtctaaagaaagcgcatgcacacatgcatacacatgcaaacacgtacacatgtacctggaacttagctgtacagtgtccttagactaagctgcctggaaagtagagagttagtgcactgtataggctcagactcttcatccacattttcctaaaaactgcaggacttcaagtcccacaattccccacatatgcacacacacatctttttgactcaagtcaaaggaaactaatttccaagttaaacaaagttggctgcattttcatcaaactatcattataccatttctcccaaatttcaaatttggctagcaatggcaaaagcatttgatcttaataactacagtgacatgtacagtgagagaggaaagaaaaagagatctttgaaagctagtagcttaggcattgaccttgataatttcttcattctcagctatactttcaagtagtgcacttaggcttgactgtgtaccaacactgcaaatgtacatgaaccctgtagatcactttgatttggcctggtgaaagaattgagggactttcataatttcacaagagcttagataagaagtccccaccccccccatcccccgctgtgaacacggagagcagtgttccattaatgtgttatccatacacctctctttttgatttttcactttttaccttccatcacagttacttaggcagtccatagacattcactggacagatacagaaataaacacataaataaactgcctatagaacagcatctgcatttaaggggagattagactctgtgcacatcaaactttacacaattttatgctttaaatggtattaaagaagttttacaatgttgcagaaactctggaatacttatccaagcaagttaaattgcaactgatctttgtttcatgttactctctgttggtggtctttggccattggaaattatcacatatttcgagagaaatgaattaacaggtaaagttattaatactaaggaaatctcttgagcaaatgggtcagtgtttcagatctgtgagtattctttcttcagatgaaaacatatccccatgcccatttcatggccctttaatggaaatgagcaactgtcttttaatgagatctttc

SEQ ID NO: 3 - protein, 338 aa, Canis familiaris
MKAARFVMRSAGSLSAASVVPREVELFSRYSPSPLSMKQLLDFDTLIKVRNRHYNVVPTMAQGIIEYKDGCTVDPVTNQNLQYFLDRFYMNRISTRMLMNQHILIFSDSETGNPTHIGSIDPSCDVAAVVQDAFECSKMLCDQYYLTSPELKLTQVNGKFPGQPIHIVYVPSHLHHMLFELFKNAMRATVEHQENWPSLTPIEVIVVLGKEDLTIKISDRGGGVPLRIIDRLFSYTYSTAPTPVMDNSRNAPLAGFGYGLPISRLYAKYFQGDLNLYSMSGYGTDAVIYLKALSSESVEKLPVFNKSAFKHYQMSIEADDWCIPSKEPKNLAKGKVAL

(19) United States
(12) Patent Application Publication
(10) Pub. No.: US 2011/0307965 A1
(43) Pub. Date: Dec. 15, 2011

(54) METHODS AND COMPOSITIONS FOR DETECTING CANINE DILATED CARDIOMYOPATHY (DCM)

(75) Inventor: Kathryn M. Meurs, Raleigh, NC (US)

(73) Assignee: Washington State University Research Foundation

(21) Appl. No.: 13/154,871

(22) Filed: Jun. 7, 2011

Related U.S. Application Data
(60) Provisional application No. 61/353,124, filed on Jun. 9, 2010.

(51) Int. Cl. A01K 67/02; C12Q 1/48; C12Q 1/68

(52) U.S. Cl. 800/8; 435/6.18; 435/15; 435/6.13

(57) ABSTRACT

This invention provides the identification of a 16 base pair deletion in a splice site region between exons 10 and 11 of the dog PDK4 gene that is linked to dilated cardiomyopathy (DCM). Also provided are methods for detecting DCM and methods of breeding dogs to reduce the prevalence of DCM.

### CROSS REFERENCE TO RELATED APPLICATION

This application claims priority to and the benefit of U.S. Provisional Application No. 61/353,124 filed Jun. 9, 2010, which is incorporated by reference herein in its entirety.

### FIELD

This disclosure relates to methods and kits for detecting dilated cardiomyopathy (DCM) in a canine subject based on detecting a deletion in the pyruvate dehydrogenase kinase 4 (PDK4) gene or a decrease in PDK4 gene expression. Further, it relates to methods of breeding dogs to decrease the prevalence of DCM in a dog breeding population.

### BACKGROUND

Dilated cardiomyopathy (DCM) is the second most common form of heart disease in dogs. Among the several breeds of dogs affected by DCM, the Doberman Pinscher is the most commonly affected breed in North American and DCM is recognized as a familial disease in this breed (Buchanan, in Textbook of canine and feline cardiology. 2nd ed. Fox et al., Eds., pp. 457-470, Philadelphia: WB Saunders Co, 1999; Meurs et al., J Vet Intern Med, 21:1016-1020, 2007). The Doberman Pinscher is a fairly popular breed, 15^{th} out of 164 registered breeds in the United States. In the Doberman Pinscher, DCM results in a large dilated left ventricle with decreased systolic myocardial function as diagnosed by echocardiography (FIG. 1A and FIG. 1B). Many affected Doberman Pinschers develop ventricular tachyarrhythmias (FIG. 2) and die of sudden death or congestive heart failure. Electron microscopy of myocardium from affected dogs demonstrates mitochondrial disruption (FIG. 3). DCM in the Doberman Pinscher has been shown to be inherited as an autosomal dominant trait (Meurs et al., J Vet Intern Med, 21:1016-1020, 2007 and FIG. 4).

DCM in dogs has many similarities to the human disease including clinical, pathological and familial aspects. In humans, at least 24 causative genes for familial DCM have been identified, including cytoskeletal, sarcomeric and mitochondrial genes (Osterziel et al., Herz, 30:529-534, 2005). Sixteen of these have been shown to be associated with autosomal dominant disease. Many of these genes have been excluded as candidates for the disease in the Doberman Pinscher (Meurs et al., Am J Vet Res, 69:1050-1053, 2008). Thus, there is a need to identify a genetic mutation associated with the development of canine DCM and to develop methods and kits for the detection of this disease.

### SUMMARY

Disclosed herein is the association between dilated cardiomyopathy (DCM) and a 16 base pair deletion in the canine PDK4 gene 5′ splice site region between exons 10 and 11. The disclosed deletion results in a measurable reduction in PDK4 gene expression that can be detected at the level of both transcription and translation.

Thus, disclosed herein are methods of detecting DCM in a canine subject, for example to determine if the canine has or is predisposed to DCM. In particular examples, the method includes detecting a defect in the PDK4 gene in a test sample obtained from the canine subject, wherein the defect results in reduced PDK4 expression. Detection of a defect in the PDK4 gene indicates that the canine subject has or is predisposed to DCM. The disclosure is not limited to particular methods of gene detection.

Additionally disclosed herein are methods of selectively breeding dogs to decrease the frequency of DCM in a dog population. For example, the method can include identifying dogs in a breeding population that have a predisposition to DCM by detecting a defect in the PDK4 gene, wherein the defect results in reduced PDK4 expression, and/or identifying dogs in a breeding population that do not have a predisposition to DCM by identifying dogs that do not have a defect in the PDK4 gene, or reduced PDK4 expression. Dogs identified that do not have a predisposition to DCM are selected and bred, thereby decreasing the frequency of DCM.

Also disclosed herein are methods of identifying a candidate compound for use in preventing or treating DCM in a canine subject. In particular examples the method includes contacting a population of cultured cells from a dog with DCM with a test compound. Subsequently, PDK4 gene expression is measured to determine a test level of PDK4 expression. The test level of PDK4 expression is compared with a control level of PDK4 expression (such as a control representing a normal level of PDK4 gene expression). Test compounds resulting in a test level of PDK4 expression greater than the control level of PDK4 expression are selected as a candidate for use in preventing or treating DCM in a canine subject.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a M-Mode echocardiograph from a Doberman Pinscher with DCM, note the dilated left ventricle (LV) in the affected dog.

FIG. 1B is a M-Mode echocardiograph from a Normal dog, note the smaller ventricle size and normal heart function.

FIG. 2 is an electrocardiogram from a DCM-affected Doberman Pinscher showing the ventricular tachyarrhythmias (arrows) associated with the disease.

FIG. 3 is an electron micrograph of myocardium from a DCM-affected dog demonstrating mitochondrial disruption (Black arrows demonstrate large abnormal mitochondria).

FIG. 4 shows a pedigree chart from a Doberman Pinscher family with DCM and indicates an autosomal dominant mode of inheritance. The proband is indicated by an arrow. Circles represent female dogs, squares represent male dogs. Solid black symbols represent affected animals; an I in the symbol represents dogs classified as indeterminate; solid white symbols represent unaffected animals; a question mark in the symbol represents animals not available for evaluation.

FIG. 5 shows a SNP association analysis of DCM-affected dogs showing an area of statistical significance on canine chromosome 14 between base pairs 23,698,921-23,931,510 (genomic sequence numbering is according to the University of California at Santa Cruz (UCSC) canine genome database, available on-line at: genome.ucsc.edu/cgi-bin/hgGateway).

FIG. 6A is a DNA sequence chromatogram of a normal (DCM-unaffected) dog, showing nucleotides 10,327-10,379 of the PDK4 gene sequence set forth in SEQ ID NO: 1; a black line denotes the 16 base pair region deleted in DCM-affected dogs (nucleotides 10,349-10,364 of SEQ ID NO: 1).

FIG. 6B is a DNA sequence chromatogram of a DCM-affected dog that is homozygous for the 16 base pair deletion in both alleles of the PDK4 gene, showing nucleotides 10,321-10,376 of SEQ ID NO: 2. The nucleotide sequence AAAGGTAG at positions 10,345-10,352 of SEQ ID NO: 2 is unique to affected dogs, and bridges the location of the 16 base pair deletion (the deleted sequence having been removed from between the two “G” residues).

FIG. 6C is a DNA sequence chromatogram of a region of the PDK4 gene in a dog that is heterozygous for the 16 base pair PDK4 deletion associated herein with DCM. A black line denotes the location of the 16 base pair region deleted in one strand of DNA. The chromatograms of the different alleles present in the heterozygote diverge following the deletion. Sequence divergence due to the deletion is also shown by the machine-assignment of the variable “N” at positions where the automated sequencer was unable to distinguish between peaks in the diverged wild type and mutant sequences. The locations in SEQ ID NOs: 1 and 2 of the sequence shown corresponds approximately to the sequences of the PDK4 gene shown in FIGS. 6A and 6B.

FIG. 7 is a chart showing the measurement of PDK4 transcript by real time RT-PCR in DCM-unaffected dogs (Nor) and dogs that are heterozygous (Het) or homozygous (Homo) for the 16 base pair deletion in the PDK4 gene. Arbitrary unit measurement of RNA transcript is on the Y axis. Genotype is indicated on the X axis. Note reduction in amount of transcript from DCM-unaffected (normal) to homozygous dogs.

FIG. 8 shows representative results of Western blot analysis of PDK4 protein expression detected with two different rabbit polyclonal antibodies directed to the PDK4 C terminus and N terminus (Rb PAb PDK4). Both antibodies detected a reduction of left ventricular myocardial PDK4 protein (LV MP) in both the heterozygous and homozygous dog (lanes 4 and 5) in comparison to the control (DCM-unaffected) dogs (lanes 2 and 3). Lane 1 shows a molecular weight standard.

FIG. 9A is the canine PDK4 nucleotide sequence (SEQ ID NO: 1) from the UCSC genome database (available on-line at genome.ucsc.edu/cgi-bin/hgGateway). The 16 base pair deletion between exons 10 and 11 is underlined and shown in bold. Introns are shown in lowercase letters. Exons are shown in capitalized letters.

FIG. 9B is the canine PDK4 protein sequence (SEQ ID NO: 3) from the UCSC genome database.

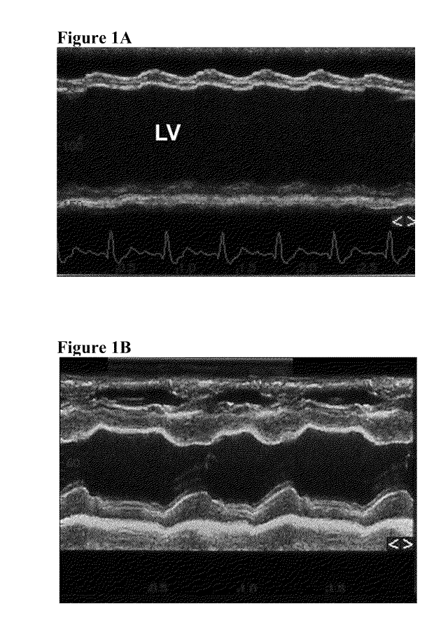

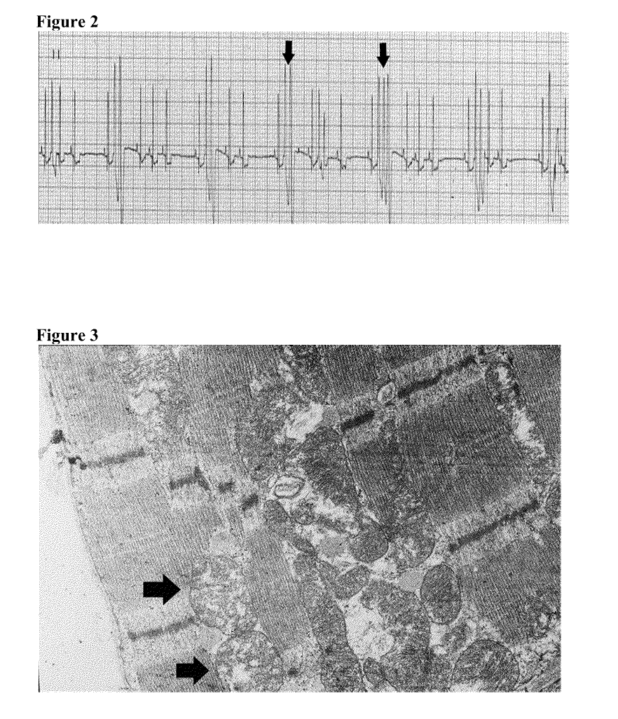

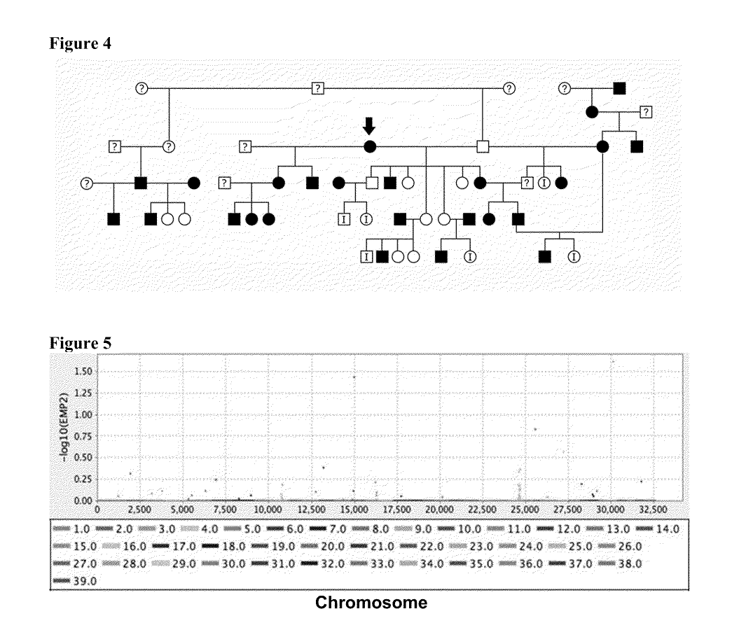

### SEQUENCE LISTING

The nucleic and/or amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleic acid sequence encoding the PDK4 gene from a wild type canine genome. Nucleotides 10,349-10,364 of this sequence are deleted in DCM-affected dogs.

SEQ ID NO: 2 is the nucleic acid sequence encoding the PDK4 gene from a DCM-affected dog that is homozygous for the 16 base pair deletion in the splice site region between PDK4 exons 10 and 11. The junction of the deleted nucleotides is at nucleotides 10,348 and 10,349.

SEQ ID NO 3 is the amino acid sequence encoding the PDK4 protein.

### DETAILED DESCRIPTION

### I. Abbreviations

ASOH allele-specific oligonucleotide hybridization

cDNA complementary DNA

DCM Dilated cardiomyopathy

HPRT hypoxanthine phosphoribosyltransferase

OLA oligonucleotide ligation assay

PCR polymerase chain reaction

PDK4 pyruvate dehydrogenase kinase 4

RT-PCR reverse transcription polymerase chain reaction

UTR untranslated region

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Altered expression: Expression of a biological molecule (for example, PDK4 RNA or PDK4 protein) in a subject or biological sample from a subject that deviates from expression if the same biological molecule in a subject or biological sample from a subject having normal or unaltered characteristics for the biological condition associated with the molecule. Aberrant expression is synonymous with altered expression. Normal expression can be found in a control, a standard for a population, etc. Altered or aberrant expression of a biological molecule may be associated with a disease (such as DCM). The term “associated with” includes an increased risk, such as a predisposition, of developing the disease as well as the disease itself. Expression may be altered in such a manner as to be increased or decreased. The directed alteration in expression of mRNA or protein may be associated with therapeutic benefits. In particular examples, altered expression of a gene, for example decreased expression of PDK4 RNA or PDK4 protein, is a result of a defect in that gene (such as a 16 by deletion in a splice site region between exons 10 and 11 of PDK4).

Altered protein expression refers to expression of a protein that is in some manner different from expression of the protein in a normal (wild type) situation. This includes but is not necessarily limited to: (1) a mutation in the protein such that one or more of the amino acid residues is different; (2) a short deletion or addition of one or a few amino acid residues to the sequence of the protein; (3) a longer deletion or addition of amino acid residues, such that an entire protein domain or sub-domain is removed or added; (4) expression of an increased amount of the protein, compared to a control or standard amount; (5) expression of an decreased amount of the protein, compared to a control or standard amount; (6) alteration of the subcellular localization or targeting of the protein; (7) alteration of the temporally regulated expression of the protein (such that the protein is expressed when it normally would not be, or alternatively is not expressed when it normally would be); and (8) alteration of the localized (for example, organ or tissue specific) expression of the protein (such that the protein is not expressed where it would normally be expressed or is expressed where it normally would not be expressed), each compared to a control or standard.

Controls or standards appropriate for comparison to a test sample, for the determination of altered expression, include samples believed to express normally as well as laboratory values, even though possibly arbitrarily set, keeping in mind that such values may vary from laboratory to laboratory. Laboratory standards and values may be set based on a known or determined population value and may be supplied in the format of a graph or table that permits easy comparison of measured, experimentally determined values.

Amplify: Increase the number or amount of a compound. To amplify a molecule or sequence of DNA is to increase the copy number of the particular DNA molecule or sequence, e.g., through an in vitro amplification technique. DNA can be amplified by any method that replicates the DNA sequence and increases the copy number of that sequence. In particular examples, DNA amplification is achieved in some embodiments using a PCR-based method including RT-PCR. Other exemplary methods of DNA amplification include isothermal amplification methods. Other representative and non-limiting examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Antibodies for use in the methods of this disclosure, for example to detect PDK4 protein, can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988). In addition, antibody fragments can also be used.

The terms bind specifically and specific binding refer to the ability of a specific binding agent (such as, an antibody) to bind to a target (such as PDK4 protein) in preference to binding to other molecular species with which the specific binding agent and target are admixed. A specific binding agent is said specifically to recognize a target when it can bind specifically to that target.

Breeding population: A population of dogs who are potentially suitable for breeding. A breeding population can be of any number of dogs and of any breed of dogs, such as the breeds disclosed herein. In one example a breeding population includes dogs that are susceptible or predisposed to DCM, such as the breeds disclosed herein.

Contacting: “Contacting” includes in solution and solid phase, for example contacting cells or other biological sample with a test compound. In one example, contacting includes contacting a population of cells isolated from a DCM-affected dog with another agent, such as a test compound.

Control: A sample or standard used for comparison with a test sample. In some embodiments, the control is a sample obtained from a healthy subject that does not have symptoms of or a predisposition to a disease such as DCM. In some embodiments, the control is a historical control or standard reference value or range of values (such as a previously tested control sample, such as a group of DCM-unaffected subjects, or group of samples that represent baseline or normal values, such as the normal level of PDK4 RNA or protein expression in a PDK4-unaffected subject). In some embodiments, the control or standard is the average PDK4 expression of DCM-unaffected dogs, regardless of dog breed. In other embodiments, the control or standard is breed-specific. Control standards and values may be set based on a known or determined population value and may be supplied in the format of a graph or table that permits easy comparison of measured, experimentally determined values.

DCM (Dilated cardiomyopathy): A familial disease that results in a large dilated left ventricle with decreased systolic myocardial function as diagnosed by echocardiography. It is the second most common form of heart disease in the dog, most commonly affecting larger-size dog breeds including Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, and Portuguese Water Dog breeds. Many affected dogs develop ventricular tachyarrhythmias and die of sudden death or congestive heart failure. Electron microscopy of myocardium from affected dogs demonstrates mitochondrial disruption. Dilated cardiomyopathy in the dog appears to be inherited as an autosomal dominant trait. DCM is commonly diagnosed through a combination of tests that may include: a physical examination, an electrocardiogram (and or ambulatory electrocardiogram), an echocardiogram (2D and M-Mode), ventricular size, post-mortem histological identification of dilated left ventricle and a history of familial disease.

Deletion: A genetic defect associated with the removal of a sequence of DNA (which may be as short as a single nucleotide), the regions on either side being joined together. A deletion in a DNA sequence is a genetic defect that can be directly detected by sequencing the region of DNA encompassing the site of deletion, thereby detecting the absence of the particular deleted sequence.

Frequency: A measure of the incidence of a disease in a population. Disease frequency is determined by any method known to the art of diagnosing the particular disease in the population. In particular examples, the frequency of DCM in a dog population can be determined by detecting the herein-described 16 base pair deletion in the PDK4 splice site region between exons 10 and 11.

Gene expression: The process by which the coded information of a nucleic acid transcriptional unit (including, for example, genomic DNA or cDNA) is converted into an operational, non-operational, or structural part of a cell, often including the synthesis of a protein. Gene expression may be measured at the RNA level or the protein level and by any method known in the art, including Northern blot, RT-PCR, Western blot, immunofluorescent or immunocytochemical protein detection, or in vitro, in situ, or in vivo protein activity assay(s).

The expression of a nucleic acid may be modulated compared to a control state, such as at a control time (for example, prior to administration of a substance or agent that affects regulation of the nucleic acid under observation) or in a control cell or subject, or as compared to another nucleic acid. Such modulation includes but is not necessarily limited to overexpression, underexpression, or suppression of expression. In addition, it is understood that modulation of nucleic acid expression may be associated with, and in fact may result in, a modulation in the expression of an encoded protein or even a protein that is not encoded by that nucleic acid.

Expression of a target gene, such as the PDK4 gene, may be measured by any method known to those of skill in the art, including for example measuring mRNA or protein levels. It is understood that a measurable reduction in gene expression is relative, and does not require absolute suppression of the gene. Thus, in certain embodiments, a measurable reduction in PDK4 gene expression requires that the gene is expressed at least 5% less than control expression levels, or at least 10% less, at least 15% less, at least 20% less, at least 25% less, or even more reduced from control levels. Thus, in some particular embodiments, a measurable reduction in PDK4 gene expression is about 30%, about 40%, about 50%, about 60%, or more reduced from control levels. In specific examples, PDK4 expression is reduced by 70%, 80%, 85%, 90%, 95%, or even more from control levels. Gene expression is substantially eliminated when expression of the gene is reduced by 90%, 95%, 98%, 99% or even 100% from control levels.

Isolated: An isolated biological component (such as a nucleic acid, peptide or protein) has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been isolated thus include nucleic acids and proteins purified by standard purification methods. The term also embraces nucleic acids, peptides and proteins prepared by recombinant expression in a host cell as well as chemically synthesized nucleic acids. The terms isolated and purified do not require absolute purity; rather, it is intended as a relative term. Thus, for example, an isolated peptide preparation is one in which the peptide or protein is more enriched than the peptide or protein is in its natural environment within a cell.

Mutation: Any change of DNA sequence, for instance within a gene or chromosome. In some instances, a mutation will alter a characteristic or trait (phenotype), but this is not always the case. A genetic defect results from a mutation (such as the herein-described 16 base pair deletion in the PDK4 splice site region between exons 10 and 11) with a deleterious phenotype. Types of mutations include base substitution point mutations (for example, transitions or transversions), deletions, and insertions. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

This term also encompasses DNA alterations that are present constitutionally, that alter the function of the encoded protein in a readily demonstrable manner, and that can be inherited by the children of an affected individual.

Gene mutations that occur outside of amino acid coding regions (e.g. untranslated or splice site regions) may also affect gene expression for example, by altering the binding sites for transcription factors on the DNA or altering proper splicing of the mRNA molecule, thereby destabilizing the RNA or shifting the frame of translation.

Nucleic acid molecule: A polymeric form of nucleotides, which may include both sense and anti-sense strands of RNA, cDNA, genomic DNA, and synthetic forms and mixed polymers thereof. A nucleotide refers to a ribonucleotide, deoxynucleotide or a modified form of either type of nucleotide. A nucleic acid molecule as used herein is synonymous with nucleic acid and polynucleotide. A nucleic acid molecule is usually at least 10 bases in length, unless otherwise specified. The term includes single- and double-stranded forms. A polynucleotide may include either or both naturally occurring and modified nucleotides linked together by naturally occurring and/or non-naturally occurring nucleotide linkages.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term nucleic acid molecule also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, the left hand end of a polynucleotide sequence written in the sense orientation is the 5′ end and the right hand end of the sequence is the 3′ end. In addition, the left hand direction of a polynucleotide sequence written in the sense orientation is referred to as the 5′ direction, while the right hand direction of the polynucleotide sequence is referred to as the 3′ direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

PDK4 gene: The pyruvate dehydrogenase kinase 4 (PDK4) gene encodes a mitochondrial protein that contributes to cardiac regulation of glucose metabolism. In particular examples, dogs with DCM have a defect in the PDK4 gene that is a 16 base pair deletion within the splice site region between exons 10 and 11 (canine genomic nomenclature previously numbered these exons as 14 and 15, respectively). As described herein, the 16 base pair deletion is a deletion in nucleotides 10,349-10,364 of SEQ ID NO: 1 and results in decreased PDK4 RNA and protein expression.

Pharmaceutical agent: A chemical compound or composition capable of inducing a desired therapeutic or prophylactic effect when properly administered to a subject or a cell. “Incubating” includes exposing an agent to a cell for a sufficient period of time for the agent to interact with a cell.

Polymerase Chain Reaction (PCR): An in vitro amplification technique that increases the number of copies of a nucleic acid molecule (for example, a nucleic acid molecule in a sample or specimen). The product of a PCR can be characterized by standard techniques known in the art, such as electrophoresis, restriction endonuclease cleavage patterns, oligonucleotide hybridization or ligation, and/or nucleic acid sequencing.

In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand). Primers can be selected that include at least 15, at least 20, at least 25, at least 30, at least 35, at least 40, at least 45, at least 50 or more consecutive nucleotides of a cancer survival factor-associated nucleotide sequence.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

Predisposition to a disease: To be susceptible and prone to a disease. A genetic defect that results in (or causes) a disease can make a subject “predisposed” to the particular disease, though the subject may not yet exhibit physical symptoms of the disease. Such a subject may become symptomatic over time or upon exposure to certain environmental or other stimulus. In particular examples, dogs carrying a genetic defect in the PDK4 gene (as described herein) that do not show symptoms of DCM (prior to mid- to late adulthood, such as prior to 6, 7, 8, 9 or more years of age) can be considered to be predisposed to DCM prior to symptom presentation.

Preventing or treating a disease: Preventing a disease refers to a therapeutic intervention that inhibits the full development of a disease, for example inhibiting the development of a large dilated left cardiac ventricle with decreased systolic myocardial function in a canine subject that is predisposed to or diagnosed with DCM. Treating a disease refers to a therapeutic intervention that ameliorates a sign or symptom of a disease or pathological condition after it has begun to develop, for example lessening the severity or frequency of ventricular tachyarrhythmias in a DCM-affected canine subject. As discussed herein, DCM is recognized in the art as an adult-onset disease that does not typically manifest before a dog is approximately 6, 7, 8, 9 or more years of age. Thus, a particular therapeutic intervention may be effective at preventing DCM if administered before full disease manifestation, but will only treat the disease if administered after symptom presentation.

Quantitative real time PCR: A method for detecting and measuring products generated during each cycle of a PCR, which products are proportionate to the amount of template nucleic acid present prior to the start of PCR. The information obtained, such as an amplification curve, can be used to quantitate the initial amounts of template nucleic acid sequence.

Reverse-transcription PCR (RT-PCR): A method for detecting, quantifying, or utilizing RNA present in a sample (such as PDK4 RNA) by a procedure wherein the RNA serves as a template for the synthesis of cDNA by a reverse transcriptase followed by PCR to amplify the cDNA. RT-PCR can be used in combination with quantitative real time PCR as a method of measuring the quantity of starting template in the reaction.

Sample (or biological sample): A biological specimen containing genomic DNA, RNA (including mRNA), protein, or combinations thereof, obtained from a subject. Examples include, but are not limited to, peripheral blood or a fraction thereof (such as serum or plasma), fine needle aspirate, urine, semen, saliva, cheek swab, tissue biopsy, surgical specimen, and autopsy material.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math. 2: 482, 1981); Needleman and Wunsch (J. Mol. Biol. 48: 443, 1970); Pearson and Lipman (PNAS. USA 85: 2444, 1988); Higgins and Sharp (Gene, 73: 237-244, 1988); Higgins and Sharp (CABIOS 5: 151-153, 1989); Corpet et al. (Nuc. Acids Res. 16: 10881-10890, 1988); Huang et al. (Comp. Appls Biosci. 8: 155-165, 1992); and Pearson et al. (Meth. Mol. Biol. 24: 307-31, 1994). Altschul et al. (Nature Genet., 6: 119-129, 1994) presents a detailed consideration of sequence alignment methods and homology calculations.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (In Molecular Cloning: A Laboratory Manual, Cold Spring Harbor, New York, 1989) and Tijssen (Laboratory Techniques in Biochemistry and Molecular Biology Part I, Ch. 2, Elsevier, New York, 1993).

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference. The following is an exemplary set of hybridization conditions:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 50% identity)

Hybridization: 6×SSC at RT to 55° C. for 16-20 hours

Wash at least twice: 2×-3×SSC at RT to 55° C. for 20-30 minutes each.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specifically hybridizable and specifically complementary are terms that indicate a sufficient degree of complementarity such that stable and specific binding occurs between an oligonucleotide (or its analog) and the DNA or RNA target, for example a probe that is specifically hybridizable to a PDK4 nucleic acid sequence found only in DCM-affected dogs (e.g. corresponding to nucleotides 10,345-10,352 of SEQ ID NO: 2). The oligonucleotide or oligonucleotide analog need not be 100% complementary to its target sequence to be specifically hybridizable, thus a PDK4 probe for detection of the 16 base pair deletion can vary in sequence so long as sufficient complementarity exists to enable hybridization of the probe to its DNA or RNA target under high stringency conditions.

Splice site region: A region of a gene encoding the intrinsic nucleic acid sequence determinants required for RNA splicing at a specific molecular location. In particular examples, the splice site region is located at the 5′-end of an intron that is removed as a result of RNA splicing, and is referred to as a 5′ splice site region. In other examples, the splice site region is located at the 3′-end of the intron, and is referred to as a 3′ splice site region. In some examples, a mutation in a splice site region can deactivate or substantially decrease RNA splicing at a particular splice site. In particular examples, a mutation in a splice site region results in the formation or use of an alternative or “cryptic” splice site that results in RNA splicing at an abnormal location.

Subject: Living multi-cellular vertebrate organism, a category that includes human and non-human mammals. In particular examples, the subject is a canine subject. In other examples, the canine subject is a particular breed of dog, such as a Doberman Pinscher, Boxer, Great Dane, Saint Bernard, Irish Wolfhound, Scottish Deerhound, Afghan Hound, Old English Sheepdog, Dalmatian, Newfoundland, Golden Retriever Greyhound, German Shepherd, English Cocker Spaniel, American Cocker Spaniel, or Portuguese Water Dog.

Under conditions sufficient for: A phrase that is used to describe any environment that permits the desired activity.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Provided herein are methods of detecting dilated cardiomyopathy (DCM) in a canine subject, for example to determine if the subject has or is predisposed to developing DCM. The methods can include detecting a defect in the PDK4 gene that results in reduced PDK4 expression in the canine subject (for example by analyzing the PDK4 gene in a sample obtained from the subject), wherein the presence of the defect indicates that the canine subject has or is predisposed to developing DCM. For example, the method can include determining whether a test sample obtained from a dog has a defect in the PDK4 gene that results in reduced PDK4 expression in the dog (for example by measuring nucleic acid expression, protein expression, by sequencing, or other method known in the art), wherein the presence of the defect indicates that the canine subject has or is predisposed to developing DCM.

In particular examples of the methods disclosed herein, detecting a defect in the PDK4 gene includes measuring PDK4 gene expression in a test sample from the canine subject, such as measuring PDK4 RNA or PDK4 protein in the test sample; and comparing the PDK4 gene expression in the test sample to a control, such as a standard reference value representing PDK4 expression in a canine subject without DCM. The presence of measurably less PDK4 expression in the test sample in comparison to the control (such as a decrease of at least 20%, at least 40%, at least 50%, at least 75%, at least 80%, at least 90%, or at least 95%) indicates that the canine subject has or is predisposed to developing DCM.

In particular examples of the described methods, the canine subject is a pure-bred Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, or Portuguese Water Dog. In other examples, the canine subject is a mixed breed, such as one containing at least 25%, at least 50% or at least 75% of one, two, three, or four of these breeds, such as a dog that is at least 25%, at least 50% or at least 75% Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, or Portuguese Water Dog.

In further examples of the disclosed methods, detecting a defect in the PDK4 gene comprises detecting a 16 base pair deletion in a 5′ splice site region between exons 10 and 11 of the PDK4 gene, wherein the 16 base pair sequence corresponds to nucleotides 10,349-10,364 of SEQ ID NO: 1, thereby detecting DCM in the canine subject. In particular examples, detecting a 16 base pair deletion in the 5′ splice site region between exons 10 and 11 of the PDK4 gene comprises isolating DNA from the canine subject; and sequencing a region of the DNA including the nucleotide sequence corresponding to nucleotides 10,349-10,364 of SEQ ID NO: 1. In other examples, detecting the 16 base pair deletion further includes amplifying the region of the DNA including the nucleotide sequence corresponding to nucleotides 10,349-10,364 of SEQ ID NO: 1, prior to sequencing the region of the DNA.

Additionally provided herein are methods of selectively breeding dogs to decrease the frequency of DCM in a dog population. The method can include identifying dogs in a breeding population that have a predisposition to DCM by detecting a defect in the PDK4 gene that results in reduced PDK4 expression, identifying dogs in a breeding population that do not have a predisposition to DCM by determining that the dog does not have a defect in the PDK4 gene that results in reduced PDK4 expression, or both. Dogs that do not have a predisposition to DCM are selected and bred, thereby decreasing the frequency of DCM. In particular examples, the dogs in the breeding population are from a breed of dogs selected from the group consisting of: Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, and Portuguese Water Dog. In other examples, the canine subject is a mixed breed, such as one containing at least 25%, at least 50% or at least 75% of one, two, three, or four of these breeds, such as a dog that is at least 25%, at least 50% or at least 75% Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, or Portuguese Water Dog.

In particular examples of the methods of selectively breeding dogs to decrease the frequency of DCM in the population, detecting a defect in the PDK4 gene includes measuring PDK4 gene expression in test samples from the dogs in the breeding population (for example using the method described above); and comparing the PDK4 gene expression, such as PDK4 RNA or PDK4 protein, in the test samples with a control (such as one representing a normal level of PDK4 gene expression), such as a standard reference value representing a normal level of PDK4 gene expression.

In other particular examples of the methods of selectively breeding dogs to decrease the frequency of DCM in the population, detecting a defect in the PDK4 gene includes detecting a 16 base pair deletion in a 5′ splice site region between exons 10 and 11 of the PDK4 gene, wherein the 16 base pair sequence corresponds to nucleotides 10,349-10,364 of SEQ ID NO: 1. In further examples, detecting a 16 base pair deletion in the 5′ splice site region between exons 10 and 11 of the PDK4 gene includes isolating DNA from the dogs of the population; and sequencing a region of the DNA including the nucleotide sequence corresponding to nucleotides 10,349-10,364 of SEQ ID NO: 1. In still further examples, detecting a 16 base pair deletion comprises amplifying the region of the DNA encompassing the nucleotide sequence corresponding to nucleotides 10,349-10,364 of SEQ ID NO: 1, prior to sequencing the region of the DNA.

Also described herein are methods of identifying a candidate compound for use in preventing or treating DCM in a canine subject. The method can include contacting a population of cultured cells from a dog with DCM with a test compound and measuring PDK4 gene expression in the population after it is contacted with the test compound to determine a test level of PDK4 expression. The test level of PDK4 expression can be compared to a control level of PDK4 expression (such as a level of PDK4 expression before adding the test compound or a level of PDK4 expression from cells cultured from a dog without DCM); and a test compound is selected as a candidate for use in preventing or treating DCM in a canine subject if the test level of PDK4 expression is greater than the control level of PDK4 expression.

The methods disclosed herein (including detecting DCM, selectively breeding dogs to decrease the frequency of DCM in a dog population, and identifying a candidate compound for use in preventing or treating DCM in a canine subject) can also include one or more of: identifying a dog suspected of having DCM or known to be at risk for developing DCM, obtaining a biological sample from a dog, isolating nucleic acids or proteins from the sample, amplifying nucleic acids (such as PDK4 or a fragment thereof) in the sample, preparing the sample for detection of PDK4 proteins or nucleic acids, and concentrating the sample.

### IV. DCM and its Diagnosis

DCM is the second most common form of heart disease in dogs; most commonly affecting the Doberman Pinscher breed as a familial disease. Other dog breeds that are commonly affected by DCM include medium to large-sized breeds such as the Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, and Portuguese Water Dog. In dogs, the disease results in a large dilated left ventricle with decreased systolic myocardial function as diagnosed by echocardiography. Many affected dogs develop ventricular tachyarrhythmias and die of sudden death or congestive heart failure. Electron microscopy of myocardium from affected dogs demonstrates mitochondrial disruption. Dilated cardiomyopathy in dogs is inherited as an autosomal dominant trait. Prior to this disclosure, DCM was commonly diagnosed through combinations of tests including: physical examination, an electrocardiogram (and or ambulatory electrocardiogram), an echocardiogram (2D and M-Mode), ventricular size, post-mortem histological identification of dilated left ventricle and available family history of DCM.

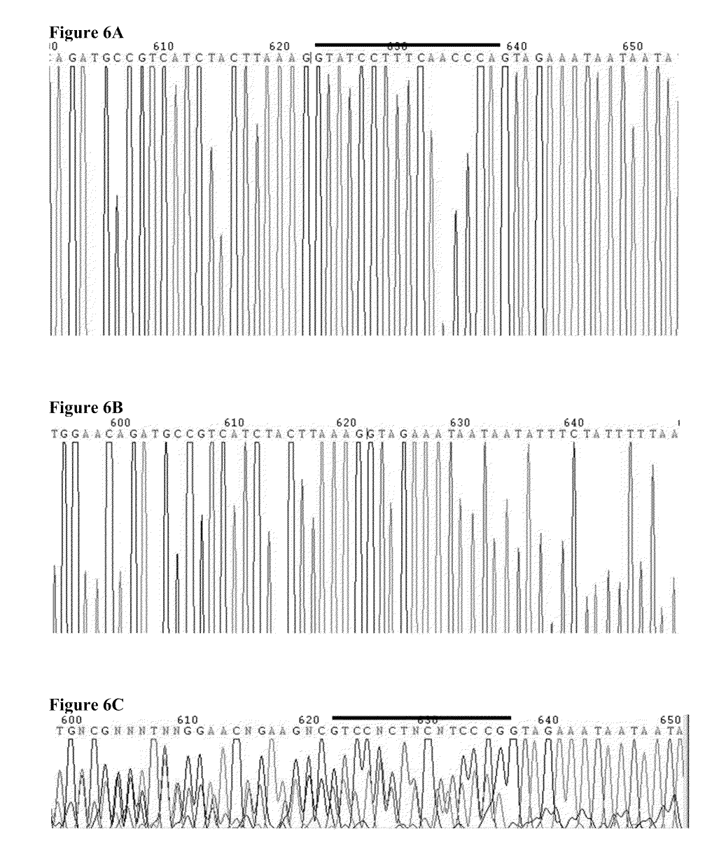

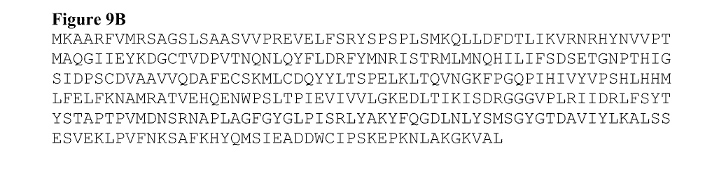

Disclosed herein is the unexpected finding that DCM results from a 16 base pair deletion in a splice site region between exons 10 and 11 of the PDK4 gene (canine genomic nomenclature formerly labeled these exons 14 and 15, respectively). PDK4 encodes a mitochondrial protein located on the canine chromosome 14. As shown in FIGS. 6A-6C, this deletion is specific to DCM-affected dogs including Dobermans. It was not observed in more than 100 unaffected dogs of ten other breeds. Although the deletion was observed in some apparently unaffected Doberman Pinschers, their health status likely reflects a later onset or variable penetrance of the disease as is observed in human beings with DCM. As shown herein, the 16 base pair deletion in a splice site region between exons 10 and 11 of the PDK4 gene results in a measurable reduction in PDK4 gene expression in the DCM-affected dogs. The nucleotide sequence of the canine PDK4 gene is set forth as SEQ ID NO: 1 and is shown in FIG. 9A. The 16 base pair sequence that is deleted between exons 10 and 11 of DCM-affected dogs is underlined and in bold: Introns are shown in lowercase letters. Exons are shown in capitalized letters. The amino acid sequence of the PDK4 protein is set forth as SEQ ID NO: 3 and is shown in FIG. 9B.

### V. Molecular Methods of Diagnosing DCM

The determination that DCM in Doberman dogs is caused by a measurable reduction in PDK4 expression resulting from a 16 base pair deletion in a 5′ splice site region between exons 10 and 11 of the PDK4 gene allows for molecular methods of DCM diagnosis in Dobermans and other dogs that are not reliant on DCM symptom onset. The deleted sequence, which is underlined in FIG. 9A and is indicated by the black bar over the sequence trace shown in FIG. 6A, occurs on chromosome 14 between base pairs 23,776,696-23,776,711 (nucleotides 10,349-10,364 of SEQ ID NO: 1). For reference, the entire sequence of canine chromosome 14 is incorporated herein by reference based on the publicly available sequence at GenBank Accession No. NC_{—}006596, as accessed on Jun. 9, 2010 on-line at ncbi.nlm.nih.gov/guide/. The canine genome is also available at the UCSC genome database, available on-line at genome.ucsc.edu/cgi-bin/hgGateway. In particular examples, the PDK4 nucleic acid or protein analyzed comprises a sequence having at least 80%, at least 90%, at least 95%, at least 98%, at least 99% or 100% sequence identity to SEQ ID NO: 1, 2 or 3 (for example to account for genetic variations that may occur in a population, such as those that do not result in disease such as DCM).

From the current disclosure, it will be appreciated that any method of detecting one or both of: (a) the DCM-specific 16 base pair deletion in a splice site region between exons 10 and 11 of the PDK4 gene or (b) a measurable reduction in PDK4 gene expression, will be a suitable diagnostic test for DCM in a canine subject. Moreover, it will also be understood that diagnosis of DCM in the canine subject will allow the affected canine subject to then be treated proactively with those pharmaceutical agents that minimize the cardiac dysfunction. For example, canine subjects identified as having or predisposed to DCM can be treated with therapies known to the art to improve heart function or lessen stress on an underperforming heart (e.g. by dilating blood vessels and increasing blood flow). By way of example, such therapies include administering to an affected dog one or more of digoxin, diuretics, pimobenden, beta blockers, and an angiotensin converting enzyme (ACE) inhibitor.

Additionally, information regarding a dog's DCM status (unaffected, homozygous, or heterozygous) could for instance be determined early in the life of the individual, and included on a license, medical record, pedigree, and so forth such that those dogs may then be used as pets and not for breeding.

The methods described herein are suitable for detecting DCM in any canine subject. In particular examples, the canine subject is pure-bred, in other examples, the canine subject is a mixture of breeds. In particular examples, the dog is a Doberman Pinscher, Boxer, Great Dane, Saint Bernard, Irish Wolfhound, Scottish Deerhound, Afghan Hound, Old English Sheepdog, Dalmatian, Newfoundland, Golden Retrievers, German Shepherd, English Cocker Spaniel, American Cocker Spaniel, Greyhound, Portuguese Water Dog, or a mixture of any of the foregoing breeds with another dog breed.

### A. Detection of the Deletion in a PDK4 Gene Splice Site

In one embodiment of the disclosed methods, DCM-affected dogs are identified by the detection of the 16 base pair deletion in a 5′ splice site region between exons 10 and 11 of the canine PDK4 gene. To perform a diagnostic test for the presence or absence of this 16 base pair deletion, a suitable genomic DNA-containing sample from a subject can be obtained and the DNA extracted using conventional techniques. DNA is extracted from any suitable tissue sample, for example, a blood sample, a buccal swab, a hair follicle preparation, sperm sample or a nasal aspirate is used as a source of cells to provide the DNA sample. The extracted DNA can then be amplified if needed, for example according to standard procedures.

The presence or absence of the 16 base pair deletion in a 5′ splice site region between exons 10 and 11 of the PDK4 gene can be determined by conventional methods including manual or automated fluorescent DNA sequencing, primer extension methods (Nikiforov et al., Nucl Acids Res 22:4167-4175, 1994), allele-specific PCR methods (Rust et al., Nucl. Acids Res. 6:3623-3629, 1993), RNase mismatch cleavage, single-strand conformation polymorphism (SSCP), denaturing gradient gel electrophoresis (DGGE), oligonucleotide hybridization, amplified fragment length polymorphism, and the like. Also, see the following U.S. Patents for descriptions of methods or applications of polymorphism analysis to disease prediction and/or diagnosis: U.S. Pat. Nos. 4,666,828; 4,801,531; 5,110,920; 5,268,267; and 5,387,506.

In some embodiments, sequences surrounding and overlapping the 16 base pair PDK4 gene splice site deletion can be useful for a number of gene mapping, targeting, and detection procedures. For example, genetic probes can be readily prepared for hybridization and detection of the described deletion. Such probe sequences may be greater than about 12 or more oligonucleotides in length and possess sufficient complementarity to distinguish between a wild type and a 16 base pair deletion sequence. In particular examples, the probe sequences hybridize to a target PDK4 sequence, such as a sequence specific to DCM-affected dogs, under high stringency conditions. In other examples, the probe sequence hybridizes to a target sequence under very high stringency conditions. Similarly, sequences surrounding and overlapping the specifically disclosed deletion can be utilized in allele-specific hybridization procedures.

In some embodiments, the deletion in a splice site region between exons 10 and 11 of the PDK4 gene can be detected by allele-specific oligonucleotide hybridization (ASOH) (Stoneking et al., Am. J. Hum. Genet. 48:370-382, 1991), which involves hybridization of labeled oligonucleotide probes to the sequence, stringent washing, and signal detection. In other embodiments, applicable methods include techniques that incorporate more robust scoring of hybridization. Examples of these procedures include the ligation chain reaction (ASOH plus selective ligation and amplification), as disclosed in Wu and Wallace (Genomics 4:560-569, 1989); mini-sequencing (ASOH plus a single base extension) as discussed in Syvanen (Meth. Mol. Biol. 98:291-298, 1998); and the use of DNA chips (miniaturized ASOH with multiple oligonucleotide arrays) as disclosed in Lipshutz et al. (BioTechniques 19:442-447, 1995). Alternatively, ASOH with single- or dual-labeled probes can be merged with PCR, as in the 5′-exonuclease assay (Heid et al., Genome Res. 6:986-994, 1996), or with molecular beacons (as in Tyagi and Kramer, Nat. Biotechnol. 14:303-308, 1996).

The 16 base pair deletion in a splice site region between exons 10 and 11 of the PDK4 gene can also be detected by dynamic allele-specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tₘ) that can be readily detected.

A variety of other techniques can be used to detect the 16 base pair deletion in the canine DNA. Merely by way of example, see U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for such methods.

### B. Detection of a Measurable Reduction in PDK4 Gene Expression

In another embodiment of the disclosed methods, DCM-affected dogs are identified by detecting a measurable reduction in PDK4 gene expression in a canine subject. Such measurement can be qualitative or quantitative. Assessment of the measurable reduction in PDK4 gene expression is made in a test subject in comparison to a control, such as an absolute or relative amount of PDK4 gene expression in a normal dog or population of dogs, such as those not having or not predisposed to DCM. Gene expression can be measured by any method known to the art and at either or both the level of transcription or translation. The determination that a given dog has a measurable reduction in PDK4 gene expression indicates that the dog is affected by DCM.

PDK4 gene expression is measured in a sample from a canine subject. The sample can be any specimen from the subject containing genomic DNA, RNA (including mRNA), protein, or combinations thereof. In particular examples, the sample is peripheral blood or a fraction thereof, fine needle aspirate, urine, saliva, cheek swab, sperm, tissue biopsy, surgical specimen, and autopsy material. In particular examples the sample is muscle tissue, for example cardiac muscle or skeletal muscle. In particular examples, the method of measuring PDK4 gene expression sample includes isolating the sample from the subject. Methods of isolating specimens from a subject, such as a blood or tissue sample are well known to the art. Any method of obtaining a sample may be used in the described methods detecting PDK4 gene expression.

PDK4 gene expression in a test subject is assessed in comparison to a control. A suitable control for use in the described methods is can be any sample or standard used for comparison with the test sample. In particular examples, the control is a sample obtained from a healthy canine subject that does not have symptoms of or a predisposition to DCM. In other examples, the control is a historical control or standard reference value or range of values (such as a previously tested control sample, such as a group of DCM-unaffected subjects, or group of samples that represent baseline or normal values, such as the normal level of PDK4 RNA or protein expression in a PDK4-unaffected subject). In some embodiments, the control or standard is the average PDK4 expression of DCM-unaffected dogs, regardless of dog breed. In other embodiments, the control or standard is breed-specific. Control standards and values may be set based on a known or determined population value and may be supplied in the format of a graph or table that permits easy comparison of measured, experimentally determined values.

In particular embodiments where transcription is measured, RT-PCR or quantitative real time RT-PCR is used to measure PDK4 expression. RT-PCR primer design is well known to the art. Any primers that are capable of detecting PDK4 expression can be used in the assays described herein. In other embodiments, quantitative primer extension is used. In still other embodiments Northern blotting is used to measure PDK4 expression.

In other embodiments, translation of PDK4 is used to determine if there is a measurable reduction in PDK4 expression. In such embodiments, PDK4 translation can be measured quantitatively (e.g. by quantitative Western blot) or qualitatively (immunofluorescent or immunohistochemical labeling of PDK4 protein in a section of tissue). Therefore, antibodies specific for PDK4 can be used to routinely detect a measurable reduction in PDK4 expression. Such evaluations can be performed, for example, in lysates prepared from cells, in fresh or frozen cells, in cells that have been smeared or touched on glass slides and then either fixed and/or dried, or in cells that have been fixed, embedded (e.g., in paraffin), and then prepared as histological sections on glass slides.

Localization and/or coordination of PDK4 expression (temporally or spatially) can also be examined using known techniques, such as isolation and comparison of PDK4 from subcellular fractions, including specific organelles, or from specific cell or tissue types, or at specific time points after an experimental manipulation. Demonstration of reduced PDK4 protein levels, in comparison to such expression in a control sample (e.g., normal, as in taken from a subject not affected by DCM), would be an alternative or supplemental approach to the direct determination of the status of the PDK4 splice site by the methods outlined above and equivalents.

The availability of antibodies specific to the PDK4 protein will facilitate the detection and quantitation of cellular PDK4 by one of a number of immunoassay methods which are well known in the art and are presented in Harlow and Lane (Antibodies, A Laboratory Manual, CSHL, New York, 1988). Any standard immunoassay format (e.g., ELISA, western blot, or RIA assay) can be used to measure PDK4 polypeptide or protein levels, and to compare these with PDK4 expression levels in control, reference, cell populations.

Immunohistochemical techniques may also be utilized for PDK4 polypeptide or protein detection. For example, a tissue sample may be obtained from a subject, and a section stained for the presence of PDK4 using a PDK4 specific binding agent (e.g., anti-PDK4 antibody) and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating a PDK4 protein, a biological sample of the subject, which sample includes cellular proteins, is required. Such a biological sample may be obtained from body cells, such as those present in a tissue biopsy, surgical specimens, or autopsy material. Quantitation of PDK4 protein can be achieved by immunoassay and compared to levels of the protein found in control cells (i.e., isolated from a subject that is not affected by DCM). Detection of a measurable reduction in PDK4 protein expression is indicative of DCM in a canine subject.

### VI. Differentiation of Individuals Homozygous Versus Heterozygous for the PDK4 Splice Site Deletion

It is possible that DCM severity depends on whether a canine subject is homozygous or heterozygous for the deletion in the 16 base pair deletion in a splice site region between exons 10 and 11 of the PDK4 gene. Thus, in some examples the method includes determining whether a canine subject is homozygous or heterozygous for the PDK4 mutation. In particular examples it is possible to determine whether a canine subject is homozygous or heterozygous by determining the DNA sequence of the canine subject in a genomic region encompassing the location of the 16 base pair deletion in the PDK4 gene (i.e. encompassing nucleotides 10,349-10,364 of SEQ ID NO: 1). By way of example, the oligonucleotide ligation assay (OLA), as described at Nickerson et al. (Proc. Natl. Acad. Sci. USA 87:8923-8927, 1990), is another method for differentiation between individuals who are homozygous versus heterozygous for the deletion in the PDK4 gene splice site. As an example of the OLA assay, when carried out in microtiter plates, one well is used for the determination of the presence of the PDK4 allele that contains the 16 base pair deletion in the PDK4 gene splice site and a second well is used for the determination of the presence of the wild type PDK4 gene splice site sequence. Thus, the results for an individual who is heterozygous for the deletion will show a signal in each of the wells.

### VII. Methods of Decreasing DCM Prevalence in a Dog Population by Selection of DCM-Free Dogs

Also disclosed herein are methods of breeding dogs that utilize the described DCM diagnostic methods to decrease the incidence of DCM in a dog population. Although DCM is a familial disease, its symptoms are adult onset, occurring typically after 6, 7, 8, 9 or more years of age. Thus, by the time a dog is used for breeding purposes it may have already transmitted a PDK4 allele with the 16 base pair deletion in a splice site region between exons 10 and 11 of the PDK4 gene.

To prevent such transmission and decrease the prevalence of DCM in a dog population, those dogs that a breeder is considering for breeding purposes are screened for DCM using the diagnostic methods described herein. In some embodiments the dog breeder has the dogs screened for the presence of the mutation in a PDK4 gene splice site (e.g. the deletion in the 16 base pair sequence in the PDK4 gene encompassing nucleotides 10,349-10,364 of SEQ ID NO: 1). In other embodiments, the dog breeder will have the dogs screened for a measurable reduction in PDK4 gene expression, for example by detecting the amount of PDK4 RNA or PDK4 protein in the dogs. Those dogs that are identified as having DCM (having a PDK4 defect) are then removed from the breeding population. Those dogs that are identified as not having DCM (having a normal PDK4 gene) are selected. The selected dogs not having DCM can be bred. Thus, the deletion in the PDK4 gene splice site will not be transmitted and the prevalence of DCM will be reduced in future generations.

### VIII. Screening Methods for Identifying Candidate Compounds to Prevent or Treat DCM

Also disclosed herein are methods of screening for candidate compounds that can be used to prevent or treat DCM. As described above, the 16 base pair deletion in a splice site region between exons 10 and 11 of the PDK4 gene results in measurably less PDK4 expression in comparison to control levels (such as PDK4 levels in dogs not having or not predisposed to DCM). The deletion in the PDK4 splice site region likely disrupts normal PDK4 RNA splicing, for example, through use of a cryptic splice site produced by the deletion or loss or decrease of the efficiency of splicing of the intron between the canine PDK4 exons 10 and 11. Also as discussed above, a canine subject may be predisposed to DCM, due to the PDK4 defect, but the disease may not develop until mid- to late adulthood (i.e., 6, 7, 8, 9 or more years of age). Thus, one of skill in the art will appreciate that compounds that increase PDK4 RNA (production or stability) or protein expression can be useful for both prevention of DCM (inhibition of disease progression) or treatment of DCM (amelioration of its symptom(s)), depending on the progression and severity of DCM in a given dog, and the age of the dog that is receiving the therapeutic compound.

Compounds that increase PDK4 expression may be identified by isolating and/or culturing cells from a DCM-symptomatic or -predisposed dog and contacting a population of the isolated cells with a test compound. In particular examples, the isolated cells are derived from muscle tissue. In particular examples the isolated cells are derived from cardiac muscle. In other particular examples, the isolated cells are derived from skeletal muscle. PDK4 gene expression is measured following the cells being contacted with the test compound to determine a test level of PDK4 expression, which level is compared to the level of a control sample (e.g., a sample of cells not contacted with the test compound). PDK4 protein or RNA expression is measured by any method, for instance any of the methods described or referred to herein. Any compound that increases PDK4 expression, stability, or level compared with the control is selected for as a candidate compound that can be useful for DCM prevention or treatment. Such compounds can then be subject to additional examination, testing and development, as will be recognized by one of ordinary skill.

In particular examples, the control is the level of PDK4 in a population of the isolated cells that were not contacted with the test compound. In other examples, the control is a standard value of PDK4 expression in at least one DCM-affected dog cell (determined previously). In still other examples, the control is the level of PDK4 expression in the test population of cells before adding the test agent.

In some embodiments, multiple test compounds may be screened simultaneously by plating cells isolated from the DCM-affected dog on multiple-welled culture plates. At least one test compound is then added to each well of the plate and allowed to contact the cells from the DCM-affected dog.

### IX. Kits

This disclosure also provides kits that enable a user to diagnose DCM in a canine subject, including reagents necessary to either detect the presence of the 16 base pair mutation in a PDK4 gene splice site or to measure PDK4 gene expression. For example, such kits can include a probe that hybridizes under high stringency conditions to a nucleotide sequencing encompassing nucleotides 10,321-10,376 of SEQ ID NO: 2, which is unique to affected dogs.

Certain kits can include reagents necessary for DNA sequencing, including, but not limited to primers, DNA polymerase, dNTPs, and ddNTPs.

Other kits can include Taq polymerase and reagents necessary for quantitative real time PCR, including but not limited to, amplification primers, fluorescent label for detection of the amplified template, nucleotides, and buffers necessary to carry out quantitative real time PCR. Other kits can include reagents for polymorphism detection by the amplified fragment length polymorphism technique, including fluorescent primers, Taq polymerase, and buffers. In further examples, the kits described herein can also contain reverse transcriptase and reagents necessary to reverse transcribe and RNA template in preparation for RT-PCR.

Other kits can include materials necessary for quantitative or qualitative detection of PDK4 protein, including one or more antibodies that specifically recognize PDK4, labeled secondary antibodies that recognize PDK4-specific antibodies, and reagents for use in detection of the label on the secondary antibody.

The materials provided in such kits may be provided in any form practicable, such as suspended in an aqueous solution or as a freeze-dried or lyophilized powder, for instance. Kits according to this invention can also include instructions, usually written instructions, to assist the user in carrying out the detection and quantification methods disclosed herein. Such instructions can optionally be provided on a computer readable medium or as a link to an internet page.

The container(s) in which the reagents are supplied can be any conventional container that is capable of holding the supplied form, for instance, microfuge tubes, ampoules, or bottles. In some applications, the reagent mixtures may be provided in pre-measured single use amounts in individual, typically disposable, tubes, microtiter plates, or equivalent containers. The containers may also be compatible with a specific automated liquid handling apparatus.

The following examples are provided to illustrate certain particular features and/or embodiments. These examples should not be construed to limit the invention to the particular features or embodiments described.

### EXAMPLES

### Example 1

### Identification of an Association Between a Mutation in the PDK4 Gene Splice Site Region and DCM

This example shows the identification of a 16 base pair deletion in a splice site region between exons 10 and 11 of the PDK4 gene of Doberman dogs affected by DCM.

Adult Doberman pinschers were prospectively recruited for participation in a study to identify the molecular cause of DCM in the Doberman pinscher. The majority of dogs were evaluated with a physical examination and an echocardiogram (2D and M-Mode) to determine disease status. A small number of dogs who were not available for echocardiogram but were at least 10 years of age and were thought to be beyond the age of disease served as additional controls. Participating dogs were classified as affected or unaffected based on the following criteria. An affected status was assigned to dogs that had echocardiographic measurements of a LVIDD >4.8 cm and a FS% <20% (O'Grady et al., Proc Am Coll Vet Med; 13:298-299, 1995). Dogs were classified as unaffected if they reached the age of 10 with a ventricular size within normal limits or were asymptomatic with no evidence of a heart murmur. DNA samples were isolated from blood samples collected from all dogs.

DNA samples from 48 affected and 48 unaffected dogs were analyzed by association analysis using a Canine Genome SNP Array containing 49,663 SNP markers. SNP genotypes were obtained following the human 500K array protocol, but with a smaller hybridization volume to allow for the smaller surface area of the canine array (Karlsson et al., Nat Genet, 39:1321-1328, 2007). Case-control GWA mapping was evaluated using the PLINK toolset (available on-line at pngu.mgh.harvard.edu/˜purcell/plink/), followed by the identification of a region of homozygosity in affected individuals based on SNP genotypes (Purcell et al., Am J Hum Genet, 81:559-575, 2007). Haplotype analysis was performed with Haploview (Barrett et al., Bioinformatics, 21:263-265, 2005). Exonic and splice site regions of statistical significance were evaluated with PCR based sequencing.

Association analysis suggested an area of statistical significance on canine chromosome 14 from 23,698,921-23,931,510 (FIG. 5). The sequence of canine chromosome 14 is also available on-line at the UCSC canine genome database (genome.ucsc.edu/cgi-bin/hgGateway). Fine-mapping of 13 additional SNPs within the region identified a statistical region of interest (p=0.001) from chromosomal sequence positions 23,774,190-23,781,919. This region spanned a single gene, pyruvate dehydrogenase kinase 4 (PDK4), which is located at Chromosome 14 from positions 23,774,809-23,786,059 on the reverse strand. The canine PDK4 sequence is available online at Ensemble Accession No. ENSCAFT00000003358; uswest.ensembl.org/Canis_familiaris/Transcript/Summary?db=core; g=ENSCAFG000000 02129; r=14:23774809-23786059; t=ENSCAFT00000003358. The PDK4 gene is of potential cardiac importance and encodes a mitochondrial protein that contributes to cardiac regulation of glucose metabolism.

Exonic and splice site regions of each allele of the PDK4 gene were evaluated with PCR based sequencing using genomic DNA in nine DCM-affected and two unaffected dogs. This method identified a 16 base pair deletion (chr 14:23,776,696-23,776,711; corresponding to 10,349-10,364 of SEQ ID NO: 1) at the 5′ splice site region of intron 10 in the canine PDK4 gene in DCM-affected Doberman Pinschers. Affected dogs were either homozygous or heterozygous for the deletion (FIGS. 6B and 6C, respectively). Additional DNA sequencing of 121 Doberman Pinschers (64 affected, 64 unaffected) identified the deletion was strongly associated with DCM affected status (p=<0.0001). This deletion was not observed in any of 100 unaffected dogs of 10 other breeds. The deletion disrupts several splice site enhancer sites based on predictions obtained from the Human Splicing Factor Database, (available on-line at umd.be/HSF/), and leads to the presumed use of a cryptic splice site within the intron.

The observations described herein show that a 16 base pair deletion in a splice site region of a gene encoding PDK4, a mitochondrial protein located on chromosome 14, is associated with the development of DCM in at least some Doberman Pinscher families. This is the first identification of a cardiac gene known to have a role in the development of dilated cardiomyopathy in the dog.

### Example 2

### PDK4 Expression is Reduced in DCM-Affected Doberman Dogs

This example shows that expression of PDK4, a mitochondrial protein, is measurably reduced in DCM-affected Doberman dogs.

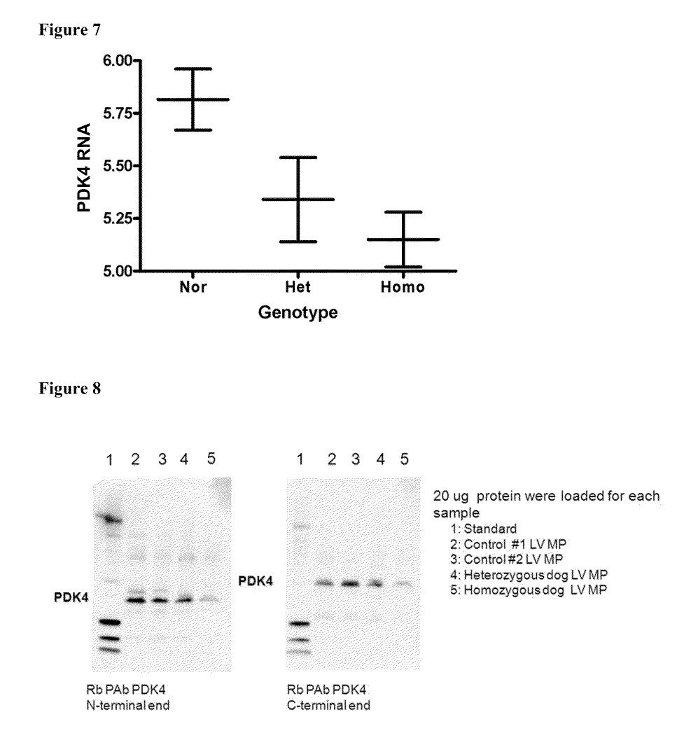

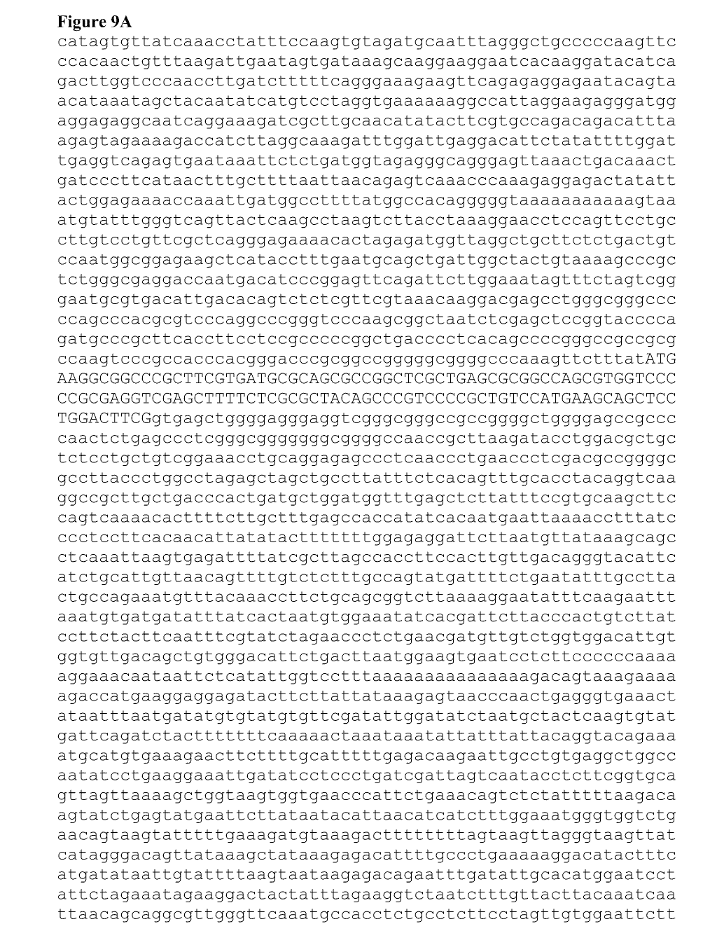

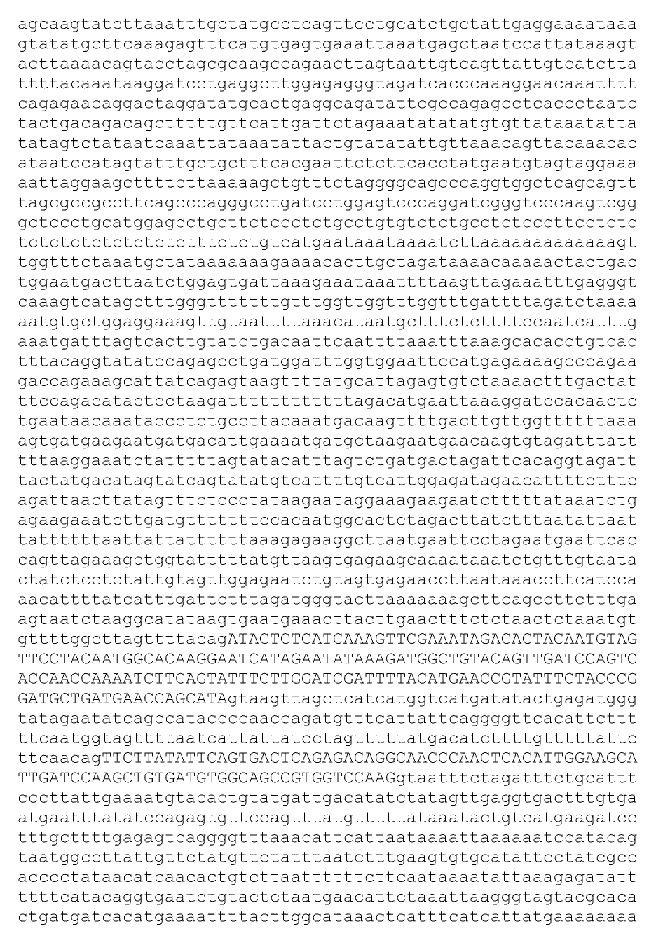

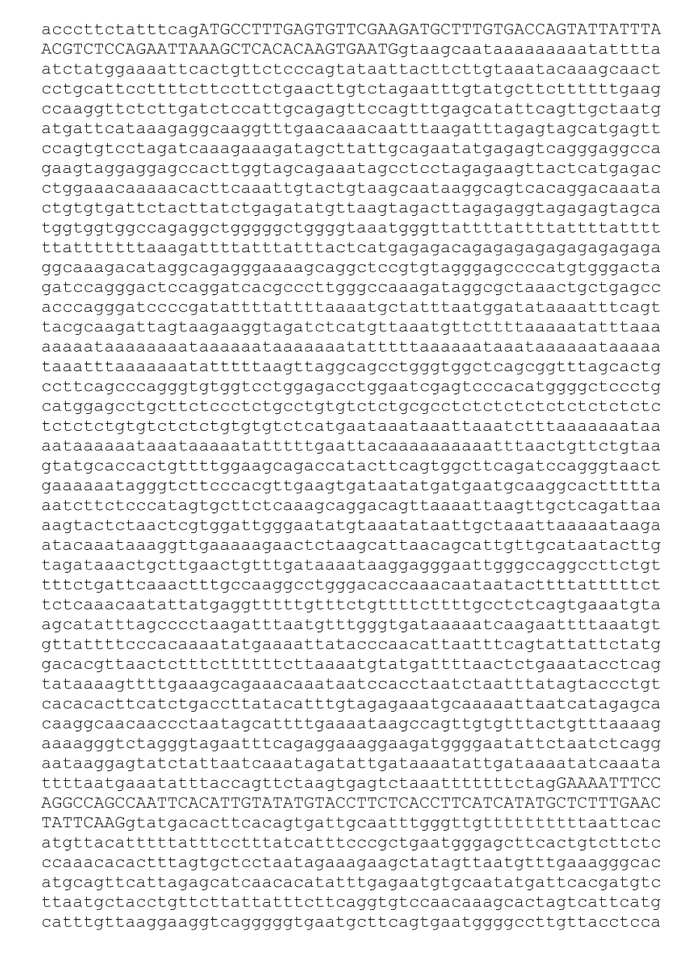

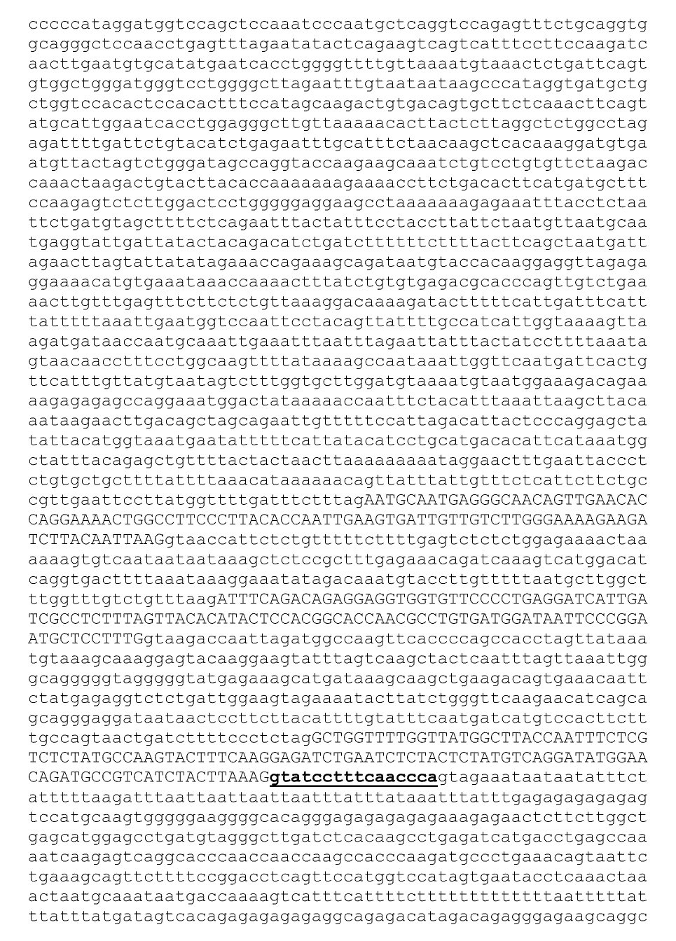

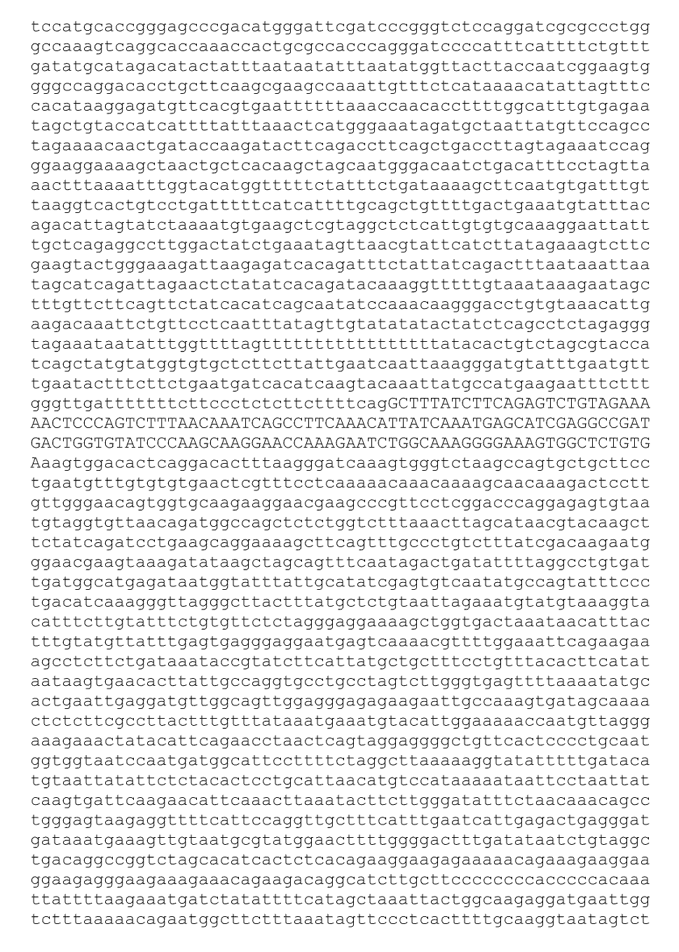

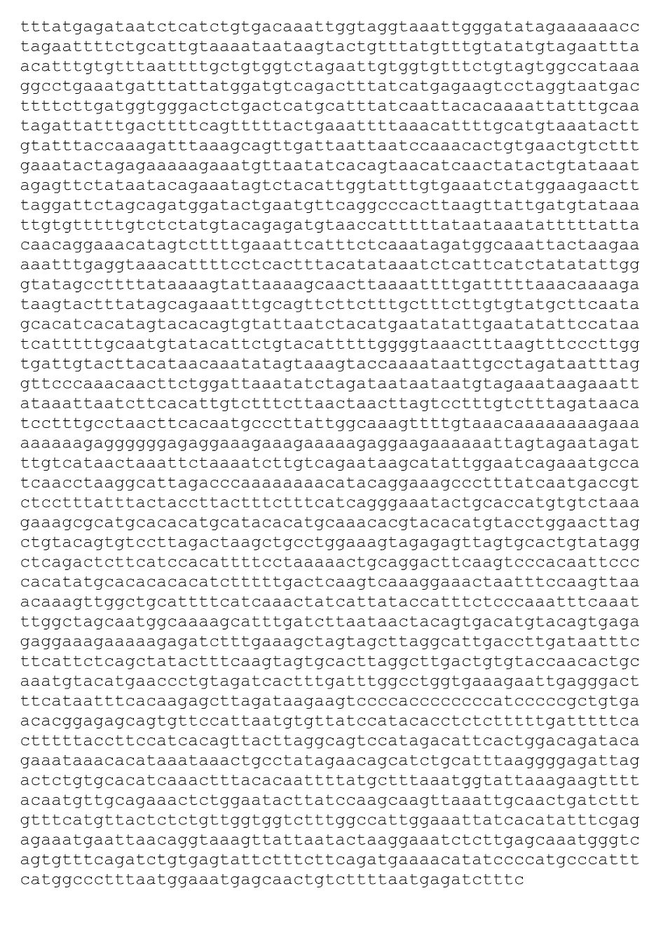

Real time PCR of the PDK4 transcript and Western blot of the protein demonstrated a reduction of RNA transcript and protein in heterozygous and homozygous DCM-affected dogs in comparison to unaffected dogs (FIGS. 7 and 8).

Real time PCR of the PDK4 transcript was performed as follows. Quantitative real-time PCR was performed with left ventricular myocardial sections from one homozygous mutant Doberman pinscher, one heterozygous Doberman pinscher, and two non-Doberman homozygous wild type controls. Approximately 50 mg of myocardium was pulverized for total RNA extraction with the RNeasy Fibrous Tissue Mini Kit (Qiagen, Valencia, Calif.). Reverse transcription was performed using Superscript II Reverse Transcriptase for cDNA synthesis (Invitrogen, Carlsbad, Calif.). Real-time PCR primers were designed for the exonic regions within PDK4 (exons 9-10). Primers were also designed for regions spanning exons 6 and 7 of the hypoxanthine phosphoribosyltransferase (HPRT) gene to be used as a housekeeping gene (TaqMan Gene Expression Assays, Applied Biosystems). SYBR® Green PCR Master Mix (Qiagen) and real-time PCR protocols were used to amplify these targets using the Applied Biosystems 7500 Fast Real-Time PCR System. Samples were evaluated in triplicate. The triplicate Ct values for each sample were averaged resulting in mean Ct values for PDK4 and HPRT. The PDK4 Ct values were standardized to the housekeeping gene by taking the difference ΔCt=Ct [Gene]−Ct [HPRT]. FIG. 7 shows a comparison of the normalized amounts of detected PDK4 transcript in dogs that have a normal, heterozygous, and homozygous genotype.

Western blot of the PDK4 protein was performed as follows: Frozen myocardial samples from left ventricular myocardial sections from one homozygous mutant Doberman pinscher, one heterozygous Doberman pinscher, and two non-Doberman homozygous wild type controls were ground to a fine powder while cooled in liquid nitrogen and homogenized in Laemmli buffer. Protein concentration was determined with the Pierce 660 protein assay (Pierce Biotechnology, Rockford, Ill.). Twenty micrograms of protein extract for each dog was separated on a 4-20% gradient polyacrylamide gel and transferred to polyvinylidene fluoride membrane. Membranes were blocked with 5% milk and PDK4 epitopes were probed (1:50 dilution) with a rabbit polyclonal antibody generated against either the N terminal region or the C terminal region of the human PDK4 polypeptide (Abcam, Cambridge, Mass.). Blots were stripped and also probed with actin monoclonal antibody (1:100) (BD Transduction Laboratories) as a loading control. The species appropriate secondary IgG-HRP (1:10,000 dilution) (1:3,000) (Santa Cruz Biotechnology Inc., Santa Cruz, Calif.) was used followed by chemiluminescence detection and optical density determination using Quantity One software (BioRad, Hercules, Calif.). Molecular weight was estimated using a standard curve generated from the Precision Protein Plus Western C standard (BioRad). Data was compared between affected and control (DCM-unaffected) animals with a T test. A p<0.05 was considered significant. FIG. 8 shows that PDK4 protein detected using either the N-terminus or C-terminus—specific antibody is measurably reduced in both heterozygous and homozygous mutant dogs in comparison to control dogs.

### Example 3

### A Method to Identify DCM-Affected Dogs by Detection of PDK4 Gene Expression

As disclosed herein, reduced PDK4 expression is indicative of DCM in canine subjects. Thus, it will be possible to identify DCM-affected dogs by comparison of PDK4 expression in a subject dog with a known range of PDK4 expression in dogs that are unaffected by DCM. As demonstrated in Example 2, the measurable reduction in PDK4 expression between DCM-affected and unaffected dogs is observable at both the level of transcription and translation. Thus, any method of evaluating PDK4 gene expression in cardiac tissue can be used.

### A. Measurement of PDK4 Transcription

PDK4 transcription can be measured quantitatively in several ways including, but not limited to RT-PCR, quantitative primer extension, or by quantitative Northern blot. To determine whether there is a measurable reduction in PDK4 transcription in the subject dog, transcription from one or more control dogs (known to be unaffected by DCM) will first be determined. Once the range of PDK4 transcription in one or more control dogs (unaffected by DCM) is determined, the level of PDK4 transcription in a subject dog can be determined. The determined amount of PDK4 transcription will then be compared to that of the control dog or dogs (known to be unaffected by DCM). A measurable reduction in PDK4 transcription in the subject dog as compared to the control dog (DCM unaffected) will indicate that the subject dog has DCM.

### B. Detection of PDK4 Protein

PDK4 expression can also be determined at the level of protein expression. Detection of protein expression can be carried out quantitatively (such as by quantitative Western blot) or qualitatively (such as by immunofluorescence). The expression of PDK4 protein will first be determined in one or more control dogs (unaffected by DCM). Then PDK4 protein expression will be determined in a subject dog. The detection in the subject dog of a measurable reduction in PDK4 protein in cardiac cells in comparison to the control dog or dogs (unaffected by DCM) will indicate that the subject dog is affected by DCM.

### Example 4

### A Method to Identify DCM-Affected Dogs by Detection of the 16 Base Pair Deletion in the PDK4 Gene Splice Site

This example demonstrates that a DCM-affected dog can be identified by detection of the 16 base pair deletion in the PDK4 gene splice site region. To screen a dog for DCM, a sample of DNA can be isolated from the dog. To determine whether the dog has a deletion in one or both copies of its PDK4 gene, a portion of the PDK4 gene inclusive of chromosome 14 base pairs 23,776,696-23,776,711 (corresponding to nucleotides 10,349-10,364 of SEQ ID NO: 1) can be amplified by PCR and then sequenced. As shown in FIGS. 6A-6C, there will be distinctive sequencing patterns found in a dog that has a wild type copy of the PDK4 gene in comparison to dogs having the 16 base pair deletion in one or both copies of the gene. From the results of the sequence analysis of the subject dog, it will be possible to diagnose the dog as having DCM.

### Example 5

### A Method of Decreasing DCM Prevalence in a Dog Population

This example demonstrates a method to decrease the incidence of DCM in dogs through screening and selective breeding. Although DCM is an inherited disease, its symptoms are adult onset. Thus, symptoms in a dog may not appear until after it has been used for breeding and has already passed on a copy of the PDK4 gene carrying the 16 base pair deletion in its gene splice site.

To decrease DCM in a dog population, candidate breeding dogs will be screened by any method of detecting DCM by either (a) a measurable reduction in PDK4 expression or (b) the 16 base pair deletion in the PDK4 gene splice site. Based on this screen, any dog that is found to be unaffected by DCM will then be deemed suitable for breeding. By breeding only those dogs that are determined to be unaffected by DCM, the incidence of DCM in the particular dog population will be decreased in future generations.

Thus, the identification of the PDK4 deletion mutation allows for the development of a breeding plan to gradually decrease the prevalence of the mutation in a dog breeding population without impacting the breed in a negative fashion through genetic screening. Pet owners and breeders will be able to test their dogs for the mutation in order to gradually remove dogs with the mutation from the breeding pool. This is particularly important since this is an adult onset disease and many dogs that have the disease will not show it until they have been used several times as a breeding animal, thus unknowingly passing the disease on to the next generation. Owners will be able to send in a small sample to test their dogs for the PDK4 mutation as young as a few weeks of age. Dogs that have the mutation can then be used as pets but would not be used as a breeding animal.

### Example 6

### Methods of Screening for Compound(s) to Prevent or Treat a DCM-Affected Subject

This example provides a method of screening for a candidate compound that can be used to prevent or treat DCM. As described herein, DCM-affected dogs have measurably less PDK4 gene expression than unaffected dogs. Thus, compounds that increase PDK4 expression will be useful for prevention or treatment of a PDK4-affected subject, for instance to inhibit DCM progression (prevention) or ameliorate DCM symptoms (treatment).

A DCM-affected dog is identified as described herein. A tissue sample, such as a cardiac tissue sample, is isolated from the dog and a cell culture established. Alternatively a previously-established culture of cells from a DCM-affected dog is used. In this illustrative example, the cells are divided and plated into eight-chamber cell culture plate(s). A different test compound is added to each of seven of the culture populations, while one of the populations is left untreated as a control. Following sufficient incubation time, the cells are collected, RNA is isolated and PDK4 expression measured by RT-PCR. The level of PDK4 RNA in the samples from the test populations is compared to that in the sample from the untreated control population. Alternatively, PDK4 protein level is tested. Any compound that produces measurably greater PDK4 expression in a test sample is then selected as a candidate compound for prevention or treatment of DCM.

In view of the many possible embodiments to which the principles of the disclosed invention may be applied, it should be recognized that the illustrated embodiments are only examples of the disclosure and should not be taken as limiting the scope of the disclosure. Rather, the scope of the invention is defined by the following claims. I therefore claim as my invention all that comes within the scope and spirit of these claims.

I claim:

1. A method of detecting dilated cardiomyopathy (DCM) in a canine subject, comprising detecting a defect in the PDK4 gene in a test sample obtained from the canine subject, wherein the defect results in reduced PDK4 expression, and wherein the presence of a defect in the PDK4 gene indicates that the canine subject has or is predisposed to DCM.

2. The method of claim 1, wherein detecting a defect in the PDK4 gene comprises:
measuring PDK4 gene expression in the test sample from the canine subject; and
comparing the PDK4 gene expression in the test sample to a control representing a normal level of PDK4 gene expression;
wherein measurably less PDK4 expression in the test sample in comparison to the control indicates that the canine subject has or is predisposed to dilated cardiomyopathy (DCM).

3. The method of claim 1, wherein the canine subject is a dog of a breed selected from the group consisting of: Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, and Portuguese Water Dog.

4. The method of claim 2, wherein measuring PDK4 gene expression comprises measuring PDK4 RNA in the test sample.

5. The method of claim 2, wherein measuring PDK4 gene expression comprises measuring PDK4 protein in the test sample.

6. The method of claim 2, wherein the control is a standard reference value.

7. The method of claim 1, wherein detecting a defect in the PDK4 gene comprises detecting a 16 base pair deletion in a 5′ splice site region between exons 10 and 11 of the PDK4 gene, wherein the 16 base pair sequence corresponds to nucleotides 10,349-10,364 of SEQ ID NO: 1, thereby detecting dilated cardiomyopathy (DCM) in the canine subject.

8. The method of claim 7, wherein detecting a 16 base pair deletion in the 5′ splice site region between exons 10 and 11 of the PDK4 gene comprises:
isolating DNA from the canine subject; and
sequencing a region of the DNA including the nucleotide sequence corresponding to nucleotides 10,349-10,364 of SEQ ID NO: 1.

9. The method of claim 8, further comprising amplifying the region of the DNA including the nucleotide sequence corresponding to nucleotides 10,349-10,364 of SEQ ID NO: 1, prior to sequencing the region of the DNA.

10. A method of selectively breeding dogs to decrease the frequency of dilated cardiomyopathy (DCM) in a dog population, comprising:
identifying dogs in a breeding population that have a predisposition to DCM by detecting a defect in the PDK4 gene, wherein the defect results in reduced PDK4 expression and/or identifying dogs in a breeding population that do not have a predisposition to DCM by determining that the PDK4 gene is normal;
selecting for breeding those dogs that do not have a predisposition to DCM; and
breeding only selected dogs, thereby decreasing the frequency of DCM in the resulting dog population.

11. The method of claim 10, wherein the dogs in the breeding population are from a breed of dogs selected from the group consisting of: Doberman Pinscher, Boxer, Great Dane, Saint Bernard, Irish Wolfhound, Scottish Deerhound, Afghan Hound, Old English Sheepdog, Dalmatian, Newfoundland, Golden Retriever, German Shepherd, English Cocker Spaniel, American Cocker Spaniel, Greyhound, and Portuguese Water Dog.

12. The method of claim 10, wherein detecting a defect in the PDK4 gene comprises:
measuring PDK4 gene expression in test samples from the dogs in the breeding population; and
comparing the PDK4 gene expression in the test samples with a control representing a normal level of PDK4 gene expression.

13. The method of claim 12, wherein measuring PDK4 gene expression comprises measuring PDK4 RNA in the test samples.

14. The method of claim 12, wherein measuring PDK4 gene expression comprises measuring PDK4 protein in the test samples.

15. The method of claim 12, wherein the control is a standard reference value.

16. The method of claim 10, wherein detecting a defect in the PDK4 gene comprises detecting a 16 base pair deletion in a 5′ splice site region between exons 10 and 11 of the PDK4 gene, wherein the 16 base pair sequence corresponds to nucleotides 10,349-10,364 of SEQ ID NO: 1.

17. The method of claim 16, wherein detecting a 16 base pair deletion in a 5′ splice site region between exons 10 and 11 of the PDK4 gene comprises
isolating DNA from the dogs of the population; and
sequencing a region of the DNA including the nucleotide sequence corresponding to nucleotides 10,349-10,364 of SEQ ID NO: 1.

18. The method of claim 17, further comprising amplifying the region of the DNA encompassing the nucleotide sequence corresponding to nucleotides 10,349-10,364 of SEQ ID NO: 1, prior to sequencing the region of the DNA.

19. A method of identifying a candidate compound for use in preventing or treating dilated cardiomyopathy (DCM) in a canine subject, comprising:
contacting cultured cells from a dog with DCM with a test compound;
measuring PDK4 gene expression in the cultured cells after contact with the test compound to determine a test level of PDK4 expression;
comparing a control level of PDK4 expression representing a normal level of PDK4 gene expression with the test level of PDK4 expression; and
selecting the test compound as a candidate for use in preventing or treating DCM in a canine subject if the test level of PDK4 expression is greater than the control level of PDK4 expression.
